(19) United States
(12) Patent Application Publication
(10) Pub. No.: US 2026/0055150 A1
(43) Pub. Date: Feb. 26, 2026

(54) GRAIL-1 PEPTIDE PRODUCTS AND METHODS

(71) Applicant: REGENTS OF THE UNIVERSITY OF MICHIGAN, Ann Arbor, MI (US)

(72) Inventors: Thomas D. Wang, Ann Arbor, MI (US); Paramita Ray, Ann Arbor, MI (US); Sangeeta Jaiswal, Ann Arbor, MI (US); Derek Nancarrow, Ann Arbor, MI (US); David G. Beer, Ann Arbor, MI (US); Dipankar Ray, Ann Arbor, MI (US)

(21) Appl. No.: 19/100,079

(22) PCT Filed: Aug. 3, 2023
(86) PCT No.: PCT/US23/71587

§ 371 Date: Jan. 30, 2025

Related U.S. Application Data
(60) Provisional application No. 63/395,280, filed on Aug. 4, 2022.

(51) Int. Cl. C07K 14/47; A61K 38/00; A61K 49/00; A61P 35/00

(52) U.S. Cl. CPC C07K 14/4702; A61K 49/0032; A61K 49/0056; A61P 35/00; A61K 38/00; C07K 2319/10

(57) ABSTRACT

The disclosure relates to GRAIL-1 peptide products and methods to target mutant p53 for treating p53-mutant related disease conditions such as Barrett's esophagus (BE) and esophageal adenocarcinoma (EAC). The disclosure also relates to methods to monitor the therapeutic response of treated BE and EAC patients by detecting expression of epidermal growth factor receptor (EGER) on BE and EAC cells.

This application claims the benefit of the filing date of U.S. Provisional Patent Application No. 63/395,280 filed Aug. 4, 2022, which is incorporated by reference herein in its entirety.

### GOVERNMENT SUPPORT

This invention was made with government support under CA-215596 and CA163059 awarded by the National Institutes of Health. The government has certain rights in the invention.

### FIELD

The disclosure relates to GRAIL-1 peptide products and methods to target mutant p53 for treating p53 mutant-related disease conditions such as Barrett's esophagus (BE) and esophageal adenocarcinoma (EAC). The disclosure also relates to methods to monitor the therapeutic response of treated BE and EAC patients by detecting expression of epidermal growth factor receptor (EGFR) on BE and EAC cells.

### INCORPORATION BY REFERENCE OF THE SEQUENCE LISTING

This application contains, as a separate part of disclosure, a Sequence Listing in computer-readable form (filename: 58214_SeqListing.xml; 39,794 bytes-XML file; created Jul. 31, 2023) which is incorporated by reference herein in its entirety.

### BACKGROUND

The tumor suppressor p53, encoded by the TP53 gene (or Trp53 gene in mice), is critical for normal cell growth and tumor prevention. The p53 tumor suppressor provides a major barrier to neoplastic transformation and tumor progression by its unique ability to act as an extremely sensitive collector of stress inputs, and to coordinate a complex framework of diverse effector pathways and processes that protect cellular homeostasis and genome stability. Missense mutations in the TP53 gene are extremely widespread in human cancers and give rise to mutant p53 proteins that lose tumor suppressive activities, and some of which exert trans-dominant repression over the wild-type counterpart. Cancer cells acquire selective advantages by retaining mutant forms of the protein, which radically subvert the nature of the p53 pathway by promoting invasion, metastasis and chemoresistance.

The development of esophageal adenocarcinoma (EAC) is a multi-decade process characterized by genomic instability. EAC patients are treated with neoadjuvant therapy followed by surgery as standard of care for local regional disease. However, only <20% achieve a complete pathological response demonstrating an unmet clinical need. Frequent (>70%) and early accumulation of mutations in the TP53 tumor suppressor gene have been reported to be key drivers both during Barrett's esophagus (BE) progression to EAC and subsequent survival/therapy resistance [The Cancer Genome Atlas Research Network, Nature, 541 (7636): 169-175 (2017); Dulak et al., Nature Genetics, 45 (5): 478-486 (2013); Secrier et al., Nature Genetics, 48 (10): 1131-1141 (2016)]. Consequently, genetic ablation of mutant TP53 in dysplastic BE/EAC cells may reduce EAC cell survival [Ray et al., Gastroenterology, 158 (3): 583-597 (2020); Ray et al., Cellular and Molecular Gastroenterology and Hepatology, 13 (1): 129-149 (2021); Martinho et al., Cellular and Molecular Gastroenterology and Hepatology, 11 (2): 449-464 (2021)]. In spite of decades of efforts, efficient clinical targeting of mutant p53 still remains elusive.

Misfolded mutant p53 protein relies on Hsp40/DNAJA1 to be recognized and chaperoned to Hsp70 for refolding and maintenance [Parrales et al., Nature Cell Biology, 18 (11): 1233-1243 (2016)]. Data in the clinic, however, indicate that inhibition of Hsp70 produces toxicity [Gestwicki and Shao, The Journal of Biological Chemistry, 294 (6): 2151-2161 (2019)].

Since human and mouse esophagus are anatomically distinct (mice have a forestomach and lack goblet cells), unlike for other cancers such as lung and pancreas, there are no ideal genetically modified mouse models for EAC. There are a esophagus specific overexpression of IL-1B transgenic mouse and a rat surgical model (esophagoduodenal anastomosis) phenocopying human BE and EAC development, however, there is no mutant p53 in such models.

A number of molecular markers associated with an increased risk for developing cancer in the digestive tract have been identified. These markers can be developed for imaging to identify high-risk patients and for early cancer detection. Previously, monoclonal antibodies have been widely studied as imaging agents for cancer detection. However, their clinical use for detecting pre-cancerous tissues has been limited by long onset for binding, immune reactions, and high costs for large quantity production.

There remains a need in the art for products and methods for treating BE and EAC and monitoring treatment.

### SUMMARY

The disclosure provides innovative GRAIL-1 products and methods to target mutant p53 for treating mutant p53-related disease conditions. The GRAIL-1 products and methods can be used, for example, in treating BE and EAC, and the disclosure also provides methods to monitor the therapeutic response of such treated patients by detecting expression of epidermal growth factor receptor (EGFR) on BE and EAC cells.

While studying BE to EAC progression, the present research group identified an isoform (Iso) switch of the E3 ubiquitin ligase RNF128 (aka GRAIL-gene related to anergy in lymphocytes), where Iso2 is lost and Iso1 levels remain high, that directly acts to stabilize the mutant p53 protein [Ray et al. 2020, supra; Ray et al. 2021, supra]. That work supports targeting GRAIL Iso1 (herein “GRAIL-1”) as a novel approach to degrade mutant p53 protein (FIG. 1). Further work described herein to understand GRAIL-1 and mutant p53 novel oncogenic cooperativity, though, unexpectedly showed that GRAIL-1 mediated mutant p53 stabilization is a ubiquitin ligase independent process and instead depends on Hsp40/DNAJA1 chaperone machinery (FIG. 2). A ten amino acid long (PMCKCDILKA) peptide representing the minimal essential sequence in GRAIL-1 (located outside the ubiquitin ligase domain) capable of interacting with DNAJA1 is provided herein. A cell permeable version of the peptide has the effects of inhibiting DNAJA1-Hsp70 chaperone activity, degrading mutant p53 protein, and reducing clonogenic survival of dysplastic BE/EAC cells (FIG. 3), thus the present disclosure contemplates methods of treating BE and EAC with GRAIL-1 peptides comprising the minimal ten amino acid essential sequence identified.

The present disclosure also contemplates methods of treatment of BE and EAC with one or more GRAIL-1 peptides provided herein in combination with a statin such as simvastatin. The present research group previously reported in Ray et al. 2020, supra, that treatment of dysplastic BE and EAC cells with simvastatin causes mutant p53 degradation and inhibition of EAC tumor growth in nude mice (FIG. 7), however the GRAIL peptides provided herein and statin target DNAJA1 by distinct mechanisms. Statin reduces the production of mevalonate-5-phospate which facilitates DNAJA1 loading to misfolded mutant p53, thus causing preferential degradation of mutant p53 by compromising chaperone activity [Parrales et al., supra]. The data herein show a synergistic effect of Pep J and simvastatin on EAC cells (FIG. 6) in causing a reduction in mutant p53 levels compared to that with monotherapy.

The disclosure contemplates methods for evaluating in patients their therapeutic response to BE and EAC treatment methods provided herein. The data herein show the loss of EGFR, similar to loss of mutant p53, following Pep J and statin treatments (FIG. 5) that correlates with loss of clonogenic survival of BE/EAC cells. The present disclosure contemplates that screening for EGFR can be used not only for early detection of dysplastic BE/EAC lesions, but also for evaluation of therapeutic responses. Fluorescently-labeled peptides can be used to detect early Barrett's neoplasia in human esophagus as shown in several clinical trials [Joshi et al., Endoscopy, 43 (2): A1-A13 (2016); Chen et al., Gut, 270 (6): 1010-1013 (2021)]. A Phase 1A first-in-human study (NCT02574858, IND #127,224) was performed to demonstrate safety for a peptide (QRHKPRE labeled with the near-infrared fluorophore Cy5, herein Cy5QRH*-Cy5) specific for EGFR to be topically administrated to human subjects. This peptide reliably detects cell surface expression of EGFR in dysplastic BE/EAC lesions [Chen et al., Endoscopy, Epub ahead of print PMID: 35299273 (Jun. 20, 2022)].

Since previously there were no genetically modified mouse models (GEMM) that effectively recapitulate early Barrett's neoplasia seen in human patients, the present disclosure provides methods for generating multiple patient-derived organoids (PDOs) with known TP53 mutation status to be used as models that are useful to demonstrate the effects of Pep J/statin treatment. These organoids have been successfully implanted in the colon of immunocompromised mice to form a representative pre-clinical in vivo model system. These 3D tissues recapitulate the developmental process of adenomas in a remarkably accurate manner and provide clinically relevant target expression levels and genetic heterogeneity.

In vivo imaging using wide-field endoscopy in the murine model system (FIG. 8) is demonstrated herein.

The disclose thus provides GRAIL-1 peptides. GRAIL-1 peptides can comprise the amino acids PMCKCDILKA (amino acids 315-324 of SEQ ID NO: 1), or a peptide analog thereof that specifically binds to DNAJA1. The GRAIL-1 peptide can comprise amino acids PMCKCDILKA (SEQ ID NO: 2), or a peptide analog thereof can comprise the amino acids PMCKATPWRE (SEQ ID NO: 3), PMCKVPPWRQ (SEQ ID NO: 4) or PMCKVPPWR (SEQ ID NO: 5).

The disclosure provides GRAIL-1 peptide conjugates comprising a GRAIL-1 peptide or peptide analog. A GRAIL-1 peptide conjugate can further comprise HIV TAT amino acids GRKKRRQRRRPQ (SEQ ID NO: 21). A GRAIL-1 peptide conjugate can comprise the amino acids GRKKRRQRRRPQPMCKCDILKA (SEQ ID NO: 6), GRKKRRQRRRPQPMCKATPWRE (SEQ ID NO: 7), GRKKRRQRRRPQPMCKVPPWRQ (SEQ ID NO: 8) or GRKKRRQRRRPQPMCKVPPWR (SEQ ID NO: 9).

The GRAIL-1 peptides, peptide analogs or GRAIL-1 peptide conjugates exhibit one or more of: specifically binding DNAJA1, inhibiting DNAJA1-Hsp70 chaperone activity, degrading mutant p53 protein, and reducing clonogenic survival of dysplastic BE/EAC cells.

The disclosure provides compositions comprising a diluent (such as a pharmaceutically acceptable diluent) and a GRAIL-1 peptide, peptide analog or GRAIL-1 peptide conjugate.

The disclosure provides methods for degrading mutant p53 in a cell comprising administering to a cell a composition provided herein.

The disclosure provides methods for treating a mutant p53-related disease condition in a patient comprising administering to the patient a composition provided herein. The mutant p53-related disease condition can be, for example, Barrett's esophagus, dysplasia, esophageal cancer (such as EAC), oral cancer, nasopharyngeal cancer, laryngeal cancer, head and neck cancer, lung cancer, ovarian cancer, pancreatic cancer or colorectoral cancer.

The disclosure provides methods for monitoring the status of Barrett's esophagus (BE) or esophageal adenocarcinoma (EAC) in a patient treated with a GRAIL-1 peptide composition provided herein, wherein the method comprises administering an EGFR-specific peptide conjugate comprising a detectable label to the colon of the patient to detect EGFR expressed on the surface of BE and EAC cells in the cell. The detectable label can be detectable by optical, photoacoustic, ultrasound, positron emission tomography or magnetic resonance imaging. The detectable label can be fluorescein isothiocyanate (FITC), Cy5, Cy5.5 or IRdye800. The EGFR-specific peptide conjugate can be QRH*-Cy5 or QRH*-KSR*-IRDye800.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

FIG. 1 shows GRAIL-1 loss reduces mutant p53 levels in EAC cells reducing clonogenic survival. (A-B) Loss of mutant p53 in OE33 (a mutant p53-driven EAC cell line) reduced clonogenic survival. (C) Independent siRNAs reduced mutant p53 levels in OE33 and OE19 cells. (D) OE33 cells were more sensitive than OE19 cells

FIG. 2 shows the C-terminal end of GRAIL encoding Ring Finger (RF) domain promotes efficient mutant p53 polyubiquitination.

FIG. 3 shows a C-terminal fragment (Frag J) of GRAIL is effective in promoting mutant p53 degradation. (A) Domain structure of GRAIL showing location of RF (ubiquitin ligase domain) and C-terminal fragment (Frag J). It further shows SSGYAS motif, a phosphodegron for β-TrCP1. Frag J and a SA mutant (Frag J^{m}) were made. (B) Overexpression of Frag J/J^{m} is effective in degrading both wild type and mutant p53 in a ubiquitin ligase independent manner. (C, D) Overexpression of Frag J reduces mutant p53 half-life from >200 mins to <60 mins. (E) Overexpression of either Frag J/J^{m} in mutant p53 driven BE cell line (CpD) reduces clonogenic survival following mutant p53 degradation.

FIG. 4 shows Frag J interacts with DNAJA1 to lock DNAJA1-Hsp70 complex inhibiting chaperone activity promoting mutant p53 degradation. (A) Model showing dynamic interactions between Hsp40/DNAJA1, in which Hsp70 recognizes misfolded protein and refolds the protein to maintain protein stability. In this process, Hsp40 recognizes misfolded protein to transfer it to Hsp70. However, following transfer, Hsp40 must dissociate from Hsp70 to allow Hsp70 to catalyze chaperone activity necessary for refolding. Thus, any hindrance to this dynamic interaction may lead to inhibition of chaperone activity. (B) Using immunoprecipitation studies using anti-GFP antibody to pull down DNAJA1, we found presence of Frag J/J^{m} locks Hsp40-Hsp70 complex, which is an indirect indicator of loss of chaperone activity. (C) The disclosure contemplates that Frag J/J^{m} interacts with DNAJA1 to form a locked DNAJA1-Hsp70 complex compromising chaperone activity to promote CHIP (another ubiquitin ligase)-mediated mutant p53 degradation.

FIG. 5 shows Frag J is localized in the rough ER. (A) Immunofluorescence (IF) staining of GRAIL Iso1 showing punctate pattern and colocalization with EEA1, an endosomal marker. (B) Similar IF staining of Frag J showed fibrillar cytosolic staining colocalizing partially with a rough ER marker, PDI.

FIG. 6 shows Frag J blocks chaperone activity and causes ER stress. (A) A luciferase double mutant (Fluc-DM) needs chaperone assistance for full luciferase activity. Thus, inhibition of chaperone activity can be indirectly measured using such reporter system. The system is used here to study the effect of Frag J/J^{m} on chaperone activity. (B, C) Fluc-DM was transfected in either BE (CpA/CpD) or p53 isogenic (H460) cell lines in the presence and absence of Frag J/J^{m}. As shown, overexpression of Frag J/J^{m} reduced luciferase activity, which is independent of TP53 mutation status. (D) Using recombinant purified DNAJA1 and Hsp70, in vitro chaperone activity was measured based on ATPase activity. Relative to control (no Frag J), presence of the fragment at 1:10 ratio showed substantial decrease (p<0.001) in chaperone activity. (E) Immunoblotting of indicated samples showed reduction of Fluc-DM levels and increased levels of pelF2α, a marker for ER stress in either wild type p53 (top panels) or mutant p53 driven (bottom panels) H460 isogenic cell lines.

FIG. 7 shows screening and identification of lead GRAIL-1 peptides showing reduction of clonogenic survival of mutant p53 driven CpD cells. (A) Effect of peptides (1-13) in wild type containing CpA cells showing minimal loss of survival up to a concentration of 30 μM. (B) Similar studies in CpD cells identified Pep 1 and Pep 5 with similar IC₅₀ of about 11 μM, suggesting therapeutic potential of these two peptides and considered as our leads. In contrast, Pep 12 showed list effects on both cell lines and was used as a negative control.

FIG. 8 shows the effect of Pep 5 on different mutant p53 containing EAC cells including Flo1 (in A), OE19 (in B), and OE33 (in C). Similar data were obtained with Pep 1 (not shown).

FIG. 9 shows Pep 1 (renamed Pep J) best fits criteria of a lead peptide. (A) Predicted Pep 5 interaction with J domain of Hsp40. (B, C) Peptide pull down assay of indicated peptides with recombinant DNAJA1 showing interactions. (D) Thermal stability assay using purified DNAJA1 and indicated TAT-less peptides showing Pep 26 (TAT-less Pep 1) as a better interactor. (E) In vitro Hsp40-Hsp70 ATPase activity showing chaperone ability suggesting Pep 26 as most effect agent working in a dose-dependent manner. (F) In vivo chaperone activity as determined using Fluc-DM showing Pep 1, 5, 9 are capable of inhibiting chaperone activity.

FIG. 10 shows GRAIL-1 peptide effects on mutant p53 levels in CpD cells. CpD cells overexpressing mutant p53 were transfected with DDK-tagged p53R175H mutant. Cells were then treated with indicated peptides (20 mM for 6 h). Cell lysates were then harvested and subjected to immunoblotting showing decrease in mutant p53 levels particularly with Pep 1, 4, 5, 7, 8, and 9. Such data correlated with clonogenic survival data shown in FIG. 7.

FIG. 11 shows the scrambled peptides. A) Table shows sequence of all synthesized scrambled peptides (SEQ ID NOs: 31-40) based on Pep 1 and Pep 5. (B) Pep 5, 6, and 9 showed least ability to downregulate mutant p53. (C and D) Peptide toxicity, as measured using WST assay showed scrambled peptides (SP) 5, 6, 9 satisfy the necessary criteria and will be used for future studies.

FIG. 12 shows Pep J synergy with statin in killing mutant p53 driven CpD cells. (A) Contemplated model of action of Pep J and statin (a cholesterol lowering drug) both targeting DNAJA1 inhibiting chaperone activity.

FIG. 13 shows Patient derived organoids (PDOs) express mutant (nuclear) p53, which is reduced upon Pep J treatment. (A) Brightfield image of p17 BE/EAC organoid with intestinal like structures, confirmed by H&E and histology, with retention of p53 heterogenous cell types in vitro. (B) Enlarged view showing intense nuclear p53, suggesting the presence of mutant p53 in dysplastic organoids. Here we plan to test the efficacy of Frag A/Pep 5 in degrading mutant p53 and abolish such dysplastic cells. (C) No major effect of Pep J or scrambled (scr) peptide treatment on size of organoid growth after 72 h of treatment (in D). (E) The same time point, however, showed significant reduction in mutant p53 (nuclear) levels as quantified based on fluorescence intensity (in F) or quantified using immunoblotting (in G).

FIG. 14 shows Pep J show synergy with simvastatin. (A) Both simvastatin (simva) and Pep J reduces mutant p53 and EGFR levels in mutant p53 driven CpD and OE33 cells with no major impact on wild type p53 containing CpA cells. (B) Simvastatin and Pep J treatment show synergy on reducing clonogenic survival of mutant p53 dependent CpD cells. (C) Dose (10 nM) and time (within 24 h) dependent reduction of EGFR levels following treatment with simvastatin in OE33 cells, implicating loss of EGFR expression can be correlated with treatment response.

FIG. 15 shows immunofluorescence (IF) and multiplexed FISH to detect expression. (A) Representative immunofluorescence (IF) images of Hcc827 cell line showing gradual, time dependent decrease in EGFR expression (see ref. 50). As shown in FIG. 4, IF staining using PDOs was standardized and will be used to quantify EGFR surface expression using QRH*-Cy5 peptide following Pep J/simvastatin treatment of PDOs. (B) Representative multiplexed FISH image of a PDOs stained with different probes (LRG5, OLFM4, and DPPIV).

FIG. 16 shows simvastatin inhibits EAC tumor growth by degrading mutant p53. (A) OE33 Tumor growth curve showing simvastatin (20 mg/Kg for 5 days) treatment inhibits tumor growth in nude mice compared to DMSO (n=5 per group). (B) Relative change in tumor volume in days 21 and 7 post tumor detection showing statistically significant difference between DMSO and simvastatin treated groups. (C) Tumor cell lysates immunoblotted using indicated antibodies, showing reduced total and active (acetylated) mutant p53 levels in simvastatin-treated group compared to control.

FIG. 17 shows establishment of in vivo model of dysplastic BE/EAC by transplanting PDOs in SCID mouse colon. (A) 3D organoids (arrow) cultured in vitro. (B) White light (WL) image is collected in vivo with rigid front-view endoscope (Storz) and shows presence of a PDO implanted in mouse colon. (C) Strong fluorescence (FL) intensity is seen from the PDO (arrow) following administration of the QRH*-Cy5, specific for EGFR. (D) Histology (H&E) of transplanted organoids processed after completion of imaging confirm the presence of the PDO. (E) Immunofluorescence staining of organoids with EpCam (epithelial neoplastic marker) shows PDO growth (arrow) in mice treated either with either DMSO (control) or single dose of simvastatin (20 mg/Kg) and collected 48 hours after statin treatment. (F) Area of organoids calculated between control and simvastatin treated groups showing reduction in size (p<0.001) in statin treated group.

FIG. 18 shows In vivo confocal images of PDOs. (A) Photo of is shown of a flexible fiber-coupled side-view confocal endomicroscope. (B) White light (WL) image shows the subtle presence of a colonic PDO (arrow) implanted in the mouse rectum. (C) Fluorescence (FL) image collected after intravenous administration of the Cy5.5-labeled peptide RTSPSSR (RTS*-Cy5.5) shows strong intensity from the PDO (arrow). Confocal images show claudin-1 expression from (D) dysplasia and (E) normal colonic mucosa with subcellular resolution. Images were collected ˜1 hour post-injection of RTS*-Cy5.5. Crypt structures and individual cells can be identified. Key: crypt (arrow), tight junction (arrowhead), lumen (I), colonocyte (c), goblet cell (g), lamina propria (lp).

FIG. 19 shows ex vivo validation of in vivo model system. Immunofluorescence (IF) shows positive staining to dysplastic crypt (arrow) in PDO versus normal mouse colonic mucosa (arrowhead) for (A) anti-human cytokeratin (hCKT), (B) anti-claudin-1 (CLN1), and (C) RTS-Cy5.5. (D) Histology (H&E) confirms the presence of human colonic dysplasia from the PDA implanted adjacent to normal mouse colonic mucosa.

FIG. 20 shows the experimental model, treatment strategies, and timeline of imaging and tissue collection/analysis in Example 3.

FIG. 21 shows results of experiments with esophageal adenocarcinoma (EAC) cells. (A, B, C) Representative radiation survival curves for OE33, OE19 and Flo1 EAC cells upon Pep J treatment. (D) Radiation enhancement ratios (ER) for OE33, OE19 and Flo1 cells are plotted from three independent experiments and plotted as mean±SE. (E) Immunoblotting for p53 protein levels. Hsc70 served as a loading control.

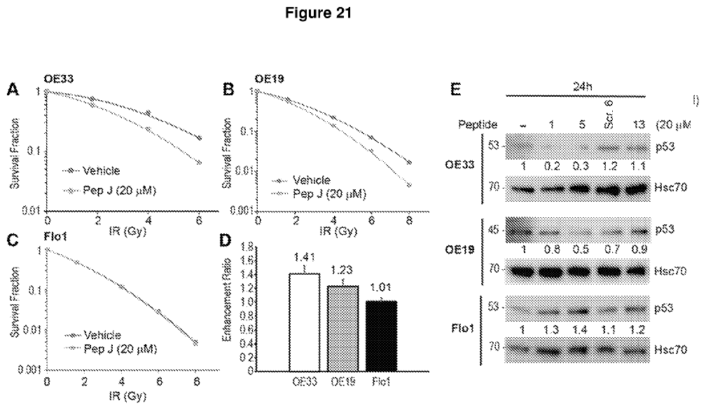

FIG. 22 shows images of effects of simvastatin, atorvastatin, and Pep J on live PDOs.

FIG. 23 shows effects of statin and Pep J on mutant p53 levels in PDOs grown in vitro. Green—p53; Red—EpCam; Blue—nucleus

FIG. 24 Barrett's esophagus (BE) organoids implanted on DSS treated mouse colon. On day 21 post implantation (p.i.), mouse was treated with DMSO/atorvastatin. Fluorescence endoscopic images were collected pre- and post-treatment using KCC.Cy5.5 peptide. Post harvest, colon was imaged with PEARL triology at 700 nm.

FIG. 25 shows confocal Z-stack images of entry of Pep J in patient derived organoids (PDOs)

### DETAILED DESCRIPTION

### Peptides

The disclosure provides “GRAIL-1 peptides” comprising:
(a) amino acids PMCKCDILKA set out in SEQ ID NO: 2 which correspond to amino acids 315-324 of SEQ ID NO: 1 (underlined below), or
(b) amino acids 315-324 of SEQ ID NO: 1 and additional amino acids of SEQ ID NO: 1 that flank amino acids 315-325, wherein the peptide comprises less than all of the C-terminal 114 amino acids of SEQ ID NO: 1.

GRAIL-1 amino acid sequence
(SEQ ID NO: 1)
MGPPPGAGVSCRGGCGFSRLLAWCFLLALSPQAPGSRGAEAVWTAYLNVSW
RVPHTGVNRTVWELSEEGVYGQDSPLEPVAGVLVPPDGPGALNACNPHTNF
TVPTVWGSTVQVSWLALIQRGGGCTFADKIHLAYERGASGAVIFNFPGTRN
EVIPMSHPGAVDIVAIMIGNLKGTKILQSIQRGIQVTMVIEVGKKHGPWVN
HYSIFFVSVSFFIITAATVGYFIFYSARRLRNARAQSRKQRQLKADAKKAI
GRLQLRTLKQGDKEIGPDGDSCAVCIELYKPNDLVRILTCNHIFHKTCVDP
WLLEHRTCPMCKCDILKALGIEVDVEDGSVSLQVPVSNEISNSASSHEEDN
RSETASSGYASVQGTDEPPLEEHVQSTNESLQLVNHEANSVAVDVIPHVDN
PTFEEDETPNQETAVREIKS

A GRAIL-1 peptide can consist of the amino acids PMCKCDILKA (SEQ ID NO: 2). Amino acids 315-325 of GRAIL-1 are demonstrated herein to be the minimal essential domain of the 114-amino acid GRAIL-1 C-terminal fragment (Frag J herein) that is outside the Ring Finger ubiquitin ligase domain and that specifically binds to DNAJA1. The GRAIL-1 peptides exhibit effects in cells including, but not limited to, one or more of: binding to DNAJA1, inhibiting DNAJA1-Hsp70 chaperone activity, degrading mutant p53 protein, and reducing clonogenic survival of dysplastic BE/EAC cells. The disclosure also provides analogs of GRAIL-1 peptides.

Other “GRAIL-1 peptides” and peptide analogs of the disclosure are Peptides 2-12 set out in Table 2, for example, Pep 4, Pep 5, Pep 7, Pep 8, and Peg 9 in Table 2.

### Peptide Conjugates

The disclosure provides peptide conjugates comprising a GRAIL-1 peptide provided herein. A “peptide conjugate” comprises at least two components, a peptide provided herein and another moiety attached to the peptide. In the GRAIL-1 peptide conjugates provided herein, the only component of the peptide conjugate that contributes to the efficacy in DNAJA1 binding, inhibiting DNAJA1-Hsp70 chaperone activity, degrading mutant p53 protein, or reducing clonogenic survival of dysplastic BE/EAC cells is the GRAIL-1 peptide. In other words, a GRAIL-1 peptide conjugate “consists essentially of” a GRAIL-1 peptide provided herein. The other moiety can comprise amino acids, but the GRAIL-1 peptide is not linked to those amino acids in nature and the other amino acids do not affect alter the efficacy of the GRAIL-1 peptide in DNAJA1 binding, inhibiting DNAJA1-Hsp70 chaperone activity, degrading mutant p53 protein, or reducing clonogenic survival of dysplastic BE/EAC cells. For example, a GRAIL-1 peptide conjugate can comprise amino acids that impart cell permeability to the GRAIL-1 peptide, such as HIV TAT amino acids GRKKRRQRRRPQ. The other amino acids can be linked to the peptides provided herein by typical peptide bonds or by other linkages known in the art. Moreover, the other moiety in a conjugate contemplated herein is not a phage in a phage display library or a component of any other type of peptide display library.

The disclosure provides methods for monitoring the status of BE and/or EAC in a patient treated with a GRAIL peptide provided herein, which methods comprise administration of an EGFR-specific peptide conjugate to the esophagus of a patient to detect EGFR expressed on the surface of BE and/or EAC cells. The EGFR-specific peptide conjugate comprises a detectable label that is detected in the methods. EGFR-specific peptide conjugates include, but are not limited to, the EGFR-specific peptide reagents disclosed in Wang et al., U.S. Pat. No. 10,500,290 (e.g., QRH*-Cy5) and the EGFR-ErbB2 heterodimeric peptide (QRH*-KSR*-IRDye800) disclosed in Chen et al. 2022, supra.

A peptide conjugate can comprise at least one detectable label as a moiety attached to a peptide provided herein. The detectable label can be detected, for example, by optical, ultrasound, PET, SPECT, or magnetic resonance imaging. The label detectable by optical imaging can be fluorescein isothiocyanate (FITC), Cy5, Cy5.5 or IRdye800 (also known as IR800CW).

A detectable label can be attached to a peptide provided herein by a peptide linker. The terminal amino acid of the linker can be a lysine such as in the exemplary linker GGGSK.

A peptide conjugate can comprise at least one therapeutic moiety attached to a peptide provided herein. The therapeutic moiety can be a chemopreventative or chemotherapeutic agent. The chemopreventative agent can be celecoxib. The chemotherapeutic agent can be carboplatin, paclitaxel, cisplatin, 5-fluorouracil (5-FU), oxaliplatin, capecitabine, chloambucil, sorafenib or irinotecan. The therapeutic moiety can be a nanoparticle or micelle encapsulating another therapeutic moiety. Carboplatin, paclitaxel, cisplatin, 5-fluorouracil (5-FU), oxaliplatin, capecitabine, chloambucil, sorafenib or irinotecan can be encapsulated.

A peptide conjugate can comprise at least one detectable label attached to the peptide or multimer form of the peptide, and at least one therapeutic moiety attached to the peptide or multimer form of the peptide.

### Compositions

The disclosure provides a composition comprising a peptide or peptide conjugate provided herein and a pharmaceutically acceptable excipient.

### Methods

The disclosure provides methods for degrading mutant p53 in a cell comprising administering to a cell a GRAIL-1 peptide or GRAIL-1 peptide conjugate provided herein. The GRAIL-1 peptide or peptide conjugate can comprise, for example, the amino acids PMCKCDILKA (SEQ ID NO: 2).

The disclosure provides methods for treating mutant p53-related disease conditions in a patient. Patients with a mutant p53-related disease condition have cells that express mutant p53 protein. Many mutant p53-related disease conditions are known in the art. Examples of such disease conditions include mutant p53-related disease conditions of the aerodigestive system. Examples of such disease conditions include mutant p53-related BE, dysplasia, esophageal cancer (squamous or adenocarcinoma), oral cancer, nasopharyngeal cancer, laryngeal cancer, head and neck cancers, lung cancer (squamous and adenocarcinoma), ovarian cancer, pancreatic cancer and colorectoral cancer. For example, the disclosure provides methods for treating BE or EAC comprising administering to a patient (e.g., a human patient) in need thereof a GRAIL-1 peptide or GRAIL-1 peptide conjugate provided herein, in which methods the GRAIL-1 peptide or GRAIL-1 peptide conjugate inhibits DNAJA1-Hsp70 chaperone activity, degrades mutant p53 protein, and/or reduces clonogenic survival of the dysplastic BE/EAC cells. The GRAIL-1 peptide or peptide conjugate can comprise, for example, the amino acids PMCKCDILKA (SEQ ID NO: 2).

The disclosure provides methods for specifically detecting BE or EAC cells in a patient comprising the steps of administering an EGFR-specific peptide conjugate provided herein comprising a detectable label to the colon of the patient and detecting binding of the EGFR-specific peptide conjugate to the cells. The detectable binding can take place in vitro, in vitro or in situ.

The phrase “specifically binds to” or “specifically detects” means that the peptide conjugate binds to and is detected in association with a type of cell, and the conjugate does not bind to and is not detected in association with another type of cell at the level of sensitivity at which the method is carried out.

Peptides or peptide conjugates and compositions thereof provided herein can be delivered by any route that effectively reaches target cells (e.g., cancer cells) in a patient including, but not limited to, administration by a topical, oral, nasal or intravenous delivery.

The disclosure provides a method of determining the effectiveness of a treatment for BE and/or EAC in a patient comprising the step of administering an EGFR-specific peptide conjugate provided herein comprising a detectable label to the patient, visualizing a first amount of cells labeled with the peptide conjugate, and comparing the first amount to a previously-visualized second amount of cells labeled with the peptide conjugate, wherein a decrease in the first amount cells labeled relative to the previously-visualized second amount of cells labeled is indicative of effective treatment. A decrease of 5% can be indicative of effective treatment. A decrease of about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or more can indicative of effective treatment. The method can further comprise obtaining a biopsy of the cells labeled by the peptide conjugate.

The disclosure provides a method for delivering a therapeutic moiety to a patient comprising the step of administering a peptide conjugate provided herein comprising the therapeutic moiety to the patient.

The disclosure provides a kit for administering a composition provided herein to a patient in need thereof, where the kit comprises a composition provided herein, instructions for use of the composition and a device for administering the composition to the patient.

### Linkers, Peptides and Peptide Analogs

As used herein, a “linker” is a sequence of amino acids located at the C-terminus of a peptide of the disclosure. The linker sequence can terminate with a lysine residue.

The presence of a linker can result in at least a 1% increase in detectable binding of an EGFR-specific peptide conjugate provided herein to BE and/or EAC cells compared to the detectable binding of the peptide conjugate in the absence of the linker. The increase in detectable binding can be at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 100-fold or more.

The term “peptide” refers to molecules of 2 to 50 amino acids, molecules of 3 to 20 amino acids, and those of 6 to 15 amino acids. Peptides and linkers contemplated herein can be 5 amino acids in length. A polypeptide or linker can be 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids in length.

Peptides include D and L forms, either purified or in a mixture of the two forms. Also contemplated by the present disclosure are peptides that compete with peptides provided herein for binding to BE and/or EAC cells.

The peptide of a peptide conjugate provided herein can be presented in multimer form. Various scaffolds are known in the art upon which multiple peptides can be presented. A peptide can be presented in multimer form on a trilysine dendritic wedge. A peptide can be presented in dimer form using an aminohexanoic acid linker. Other scaffolds known in the art include, but are not limited to, other dendrimers and polymeric (e.g., PEG) scaffolds.

It will be understood that peptides and linkers provided herein optionally incorporate modifications known in the art and that the location and number of such modifications are varied to achieve an optimal effect in the peptide and/or linker analog.

A peptide analog having a structure based on one of the peptides disclosed herein (the “parent peptide”) can differ from the parent peptide in one or more respects. Accordingly, as appreciated by one of ordinary skill in the art, the teachings regarding the parent peptides provided herein can also be applicable to the peptide analogs.

The peptide analog can comprise the structure of a parent peptide, except that the peptide analog comprises one or more non-peptide bonds in place of peptide bond(s). The peptide analog can comprise in place of a peptide bond, an ester bond, an ether bond, a thioether bond, an amide bond, and the like. The peptide analog can be a depsipeptide comprising an ester linkage in place of a peptide bond.

The peptide analog can comprise the structure of a parent peptide described herein, except that the peptide analog comprises one or more amino acid substitutions, e.g., one or more conservative amino acid substitutions. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc. Some illustrative peptide analogs provided herein are Pep 4, Pep 5 and Pep 7 set out in Table 2.

The peptide analog can comprise one or more synthetic amino acids, e.g., an amino acid non-native to a mammal. Synthetic amino acids include β-alanine (β-Ala), N-□-methyl-alanine (Me-Ala), aminobutyric acid (Abu), γ-aminobutyric acid (γ-Abu), aminohexanoic acid (ε-Ahx), aminoisobutyric acid (Aib), aminomethylpyrrole carboxylic acid, aminopiperidinecarboxylic acid, aminoserine (Ams), aminotetrahydropyran-4-carboxylic acid, arginine N-methoxy-N-methyl amide, β-aspartic acid (β-Asp), azetidine carboxylic acid, 3-(2-benzothiazolyl) alanine, α-tert-butylglycine, 2-amino-5-ureido-n-valeric acid (citrulline, Cit), β-Cyclohexylalanine (Cha), acetamidomethyl-cysteine, diaminobutanoic acid (Dab), diaminopropionic acid (Dpr), dihydroxyphenylalanine (DOPA), dimethylthiazolidine (DMTA), γ-Glutamic acid (γ Glu), homoserine (Hse), hydroxyproline (Hyp), isoleucine N-methoxy-N-methyl amide, methyl-isoleucine (Melle), isonipecotic acid (Isn), methyl-leucine (MeLeu), methyl-lysine, dimethyl-lysine, trimethyl-lysine, methanoproline, methionine-sulfoxide (Met(O)), methionine-sulfone (Met(O₂)), norleucine (Nle), methyl-norleucine (Me-Nle), norvaline (Nva), ornithine (Orn), para-aminobenzoic acid (PABA), penicillamine (Pen), methylphenylalanine (MePhe), 4-Chlorophenylalanine (Phe(4-Cl)), 4-fluorophenylalanine (Phe(4-F)), 4-nitrophenylalanine (Phe(4-NO₂)), 4-cyanophenylalanine ((Phe (4-CN)), phenylglycine (Phg), piperidinylalanine, piperidinylglycine, 3,4-dehydroproline, pyrrolidinylalanine, sarcosine (Sar), selenocysteine (Sec), O-Benzyl-phosphoserine, 4-amino-3-hydroxy-6-methylheptanoic acid (Sta), 4-amino-5-cyclohexyl-3-hydroxypentanoic acid (ACHPA), 4-amino-3-hydroxy-5-phenylpentanoic acid (AHPPA), 1,2,3,4,-tetrahydro-isoquinoline-3-carboxylic acid (Tic), tetrahydropyranglycine, thienylalanine (Thi), O-benzyl-phosphotyrosine, O-Phosphotyrosine, methoxytyrosine, ethoxytyrosine, O-(bis-dimethylamino-phosphono)-tyrosine, tyrosine sulfate tetrabutylamine, methyl-valine (MeVal), and alkylated 3-mercaptopropionic acid.

The peptide analog can comprise one or more non-conservative amino acid substitutions and the peptide analog still functions to a similar extent, the same extent, or an improved extent as the parent peptide. The peptide analog can comprise one or more non-conservative amino acid substitutions exhibits about the same or greater binding to HCC cells in comparison to the parent peptide.

The peptide analog can comprise one or more amino acid insertions or deletions, in comparison to the parent peptide described herein. The peptide analog can comprise an insertion of one or more amino acids in comparison to the parent peptide. The peptide analog can comprise a deletion of one or more amino acids in comparison to the parent peptide. The peptide analog can comprise an insertion of one or more amino acids at the N- or C-terminus in comparison to the parent peptide. The peptide analog can comprise a deletion of one or more amino acids at the N- or C-terminus in comparison to the parent peptide. In all these instances, the peptide analog still exhibits about the same or greater binding to BE and/or EAC cells.

### Detectable Markers

As used herein, a “detectable marker” is any label that can be used to identify the binding of a composition of the disclosure to HCC cells. Non-limiting examples of detectable markers are fluorophores, chemical or protein tags that enable the visualization of a polypeptide. Visualization in certain aspects is carried out with the naked eye, or a device (for example and without limitation, an endoscope) and can also involve an alternate light or energy source.

Fluorophores, chemical and protein tags that are contemplated for use herein include, but are not limited to, FITC, Cy5, Cy 5.5, Cy 7, Li-Cor, a radiolabel, biotin, luciferase, 1,8-ANS (1-Anilinonaphthalene-8-sulfonic acid), 1-Anilinonaphthalene-8-sulfonic acid (1,8-ANS), 5-(and-6)-Carboxy-2′, 7′-dichlorofluorescein pH 9.0, 5-FAM pH 9.0, 5-ROX (5-Carboxy-X-rhodamine, triethylammonium salt), 5-ROX pH 7.0, 5-TAMRA, 5-TAMRA PH 7.0, 5-TAMRA-MeOH, 6 JOE, 6,8-Difluoro-7-hydroxy-4-methylcoumarin pH 9.0, 6-Carboxyrhodamine 6G pH 7.0, 6-Carboxyrhodamine 6G, hydrochloride, 6-HEX, SE pH 9.0, 6-TET, SE pH 9.0, 7-Amino-4-methylcoumarin pH 7.0, 7-Hydroxy-4-methylcoumarin, 7-Hydroxy-4-methylcoumarin pH 9.0, Alexa 350, Alexa 405, Alexa 430, Alexa 488, Alexa 532, Alexa 546, Alexa 555, Alexa 568, Alexa 594, Alexa 647, Alexa 660, Alexa 680, Alexa 700, Alexa Fluor 430 antibody conjugate pH 7.2, Alexa Fluor 488 antibody conjugate pH 8.0, Alexa Fluor 488 hydrazide-water, Alexa Fluor 532 antibody conjugate pH 7.2, Alexa Fluor 555 antibody conjugate pH 7.2, Alexa Fluor 568 antibody conjugate pH 7.2, Alexa Fluor 610 R-phycoerythrin streptavidin pH 7.2, Alexa Fluor 647 antibody conjugate pH 7.2, Alexa Fluor 647 R-phycoerythrin streptavidin pH 7.2, Alexa Fluor 660 antibody conjugate pH 7.2, Alexa Fluor 680 antibody conjugate pH 7.2, Alexa Fluor 700 antibody conjugate pH 7.2, Allophycocyanin pH 7.5, AMCA conjugate, Amino Coumarin, APC (allophycocyanin), Atto 647, BCECF pH 5.5, BCECF pH 9.0, BFP (Blue Fluorescent Protein), Calcein, Calcein pH 9.0, Calcium Crimson, Calcium Crimson Ca2+, Calcium Green, Calcium Green-1 Ca2+, Calcium Orange, Calcium Orange Ca2+, Carboxynaphthofluorescein pH 10.0, Cascade Blue, Cascade Blue BSA pH 7.0, Cascade Yellow, Cascade Yellow antibody conjugate pH 8.0, CFDA, CFP (Cyan Fluorescent Protein), CI-NERF pH 2.5, CI-NERF pH 6.0, Citrine, Coumarin, Cy 2, Cy 3, Cy 3.5, Cy 5, C5.5, CyQUANT GR-DNA, Dansyl Cadaverine, Dansyl Cadaverine, MeOH, DAPI, DAPI-DNA, Dapoxyl (2-aminoethyl) sulfonamide, DDAO pH 9.0, Di-8 ANEPPS, Di-8-ANEPPS-lipid, Dil, DiO, DM-NERF pH 4.0, DM-NERF pH 7.0, DsRed, DTAF, dTomato, eCFP (Enhanced Cyan Fluorescent Protein), eGFP (Enhanced Green Fluorescent Protein), Eosin, Eosin antibody conjugate pH 8.0, Erythrosin-5-isothiocyanate pH 9.0, eYFP (Enhanced Yellow Fluorescent Protein), FDA, FITC antibody conjugate pH 8.0, FIASH, Fluo-3, Fluo-3 Ca2+, Fluo-4, Fluor-Ruby, Fluorescein, Fluorescein 0.1 M NaOH, Fluorescein antibody conjugate pH 8.0, Fluorescein dextran pH 8.0, Fluorescein pH 9.0, Fluoro-Emerald, FM 1-43, FM 1-43 lipid, FM 4-64, FM 4-64, 2% CHAPS, Fura Red Ca²⁺, Fura Red, high Ca, Fura Red, low Ca, Fura-2 Ca2+, Fura-2, Fura-2, GFP (S65T), HcRed, Indo-1 Ca²⁺, Indo-1, Ca free, Indo-1, Ca saturated, IDRdye800 (IR800CW), JC-1, JC-1 pH 8.2, Lissamine rhodamine, Lucifer Yellow, CH, Magnesium Green, Magnesium Green Mg2+, Magnesium Orange, Marina Blue, mBanana, mCherry, mHoneydew, mOrange, mPlum, mRFP, mStrawberry, mTangerine, NBD-X, NBD-X, MeOH, NeuroTrace 500/525, green fluorescent Nissl stain-RNA, Nile Blue, Nile Red, Nile Red-lipid, Nissl, Oregon Green 488, Oregon Green 488 antibody conjugate pH 8.0, Oregon Green 514, Oregon Green 514 antibody conjugate pH 8.0, Pacific Blue, Pacific Blue antibody conjugate pH 8.0, Phycoerythrin, R-Phycoerythrin pH 7.5, ReAsH, Resorufin, Resorufin pH 9.0, Rhod-2, Rhod-2 Ca2+, Rhodamine, Rhodamine 110, Rhodamine 110 pH 7.0, Rhodamine 123, MeOH, Rhodamine Green, Rhodamine phalloidin pH 7.0, Rhodamine Red-X antibody conjugate pH 8.0, Rhodamine Green pH 7.0, Rhodol Green antibody conjugate pH 8.0, Sapphire, SBFI-Na+, Sodium Green Na+, Sulforhodamine 101, Tetramethylrhodamine antibody conjugate pH 8.0, Tetramethylrhodamine dextran pH 7.0, and Texas Red-X antibody conjugate pH 7.2.

Non-limiting examples of chemical tags contemplated herein include radiolabels. For example and without limitation, radiolabels that contemplated in the compositions and methods of the present disclosure include ¹¹C, ¹³N, ¹⁵O, ¹⁸F, ³²P, ⁵²Fe, ⁶²Cu, ⁶⁴Cu, ⁶⁷Cu, ⁶⁷Ga, ⁶⁸Ga, ⁸⁶Y, ⁸⁹Zr, ⁹⁰Y, ⁹⁴mTc, ⁹⁴Tc, ⁹⁵Tc, ⁹⁹mTc, ¹⁰³Pd, ¹⁰⁵Rh, ¹⁰⁹Pd, ¹¹¹Ag, ¹¹¹In, ¹²³I, ¹²⁴I, ¹²⁵I, ¹³¹I, ¹⁴⁰La, ¹⁴⁹Pm, ¹⁵³Sm, ¹⁵⁴⁻¹⁵⁹Gd, ¹⁶⁵Dy, ¹⁶⁶Dy, ¹⁶⁶Ho, ¹⁶⁹Yb, ¹⁷⁵Yb, ¹⁷⁵Lu, ¹⁷⁷Lu, ¹⁸⁶Re, ¹⁸⁸Re, ¹⁹²Ir, ¹⁹⁸Au, ¹⁹⁹Au, and ²¹²Bi.

For positron emission tomography (PET) tracers including, but not limited to, carbon-11, nitrogen-13, oxygen-15 and fluorine-18 are used.

A worker of ordinary skill in the art will appreciate that there are many such detectable markers that can be used to visualize a cell, in vitro, in vitro or ex vivo.

### Therapeutic Moieties

Therapeutic moieties contemplated herein include, but are not limited to, polypeptides (including protein therapeutics) or peptides, small molecules, chemotherapeutic agents, or combinations thereof.

The term “small molecule”, as used herein, refers to a chemical compound, for instance a peptidometic or oligonucleotide that can optionally be derivatized, or any other low molecular weight organic compound, either natural or synthetic.

By “low molecular weight” is meant compounds having a molecular weight of less than 1000 Daltons, typically between 300 and 700 Daltons. Low molecular weight compounds, in various aspects, are about 100, about 150, about 200, about 250, about 300, about 350, about 400, about 450, about 500, about 550, about 600, about 650, about 700, about 750, about 800, about 850, about 900, about 1000 or more Daltons.

The therapeutic moiety can be a protein therapeutic. Protein therapeutics include, without limitation, cellular or circulating proteins as well as fragments and derivatives thereof. Still other therapeutic moieties include polynucleotides, including without limitation, protein coding polynucleotides, polynucleotides encoding regulatory polynucleotides, and/or polynucleotides which are regulatory in themselves. Optionally, the compositions comprise a combination of the compounds described herein.

Protein therapeutics can include cytokines or hematopoietic factors including without limitation IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2a, cytokine-induced neutrophil chemotactic factor 23, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor receptor, TNF, including TNF0, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

Therapeutic moieties can also include chemotherapeutic agents. A chemotherapeutic agent contemplated for use in a peptide conjugate provided herein includes, without limitation, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, capecitabine, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural conjugates including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinum coordination complexes such as oxaliplatin, cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; topoisomerase inhibitors such as irinotecan; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide. Chemotherapeutic agents such as gefitinib, sorafenib and erlotinib are also specifically contemplated.

Therapeutic moieties to be attached to a peptide described herein also include nanoparticles or micelles that, in turn, encapsulate another therapeutic moiety. The nanoparticles can be polymeric nanoparticles such as described in Zhang et al., ACS NANO, 2 (8): 1696-1709 (2008) or Zhong et al., Biomacromolecules, 15:1955-1969 (2014). The micelles can be polymeric micelles such as octadecyl lithocholate micelles described in Khondee et al., J. Controlled Release, 199:114-121 (2015) and WO 2017/096076 (published Jun. 8, 2017). The peptide conjugates comprising nanoparticles or micelles can encapsulate, for example, carboplatin, paclitaxel, cisplatin, 5-fluorouracil (5-FU), oxaliplatin, capecitabine or irinotecan.

### Dosages

Dosages of a peptide or peptide conjugate provided herein are administered as a dose measured in, for example, mg/kg. Contemplated mg/kg doses include, but are not limited to, about 1 mg/kg to about 60 mg/kg. Illustrative specific ranges of doses in mg/kg include about 1 mg/kg to about 20 mg/kg, about 5 mg/kg to about 20 mg/kg, about 10 mg/kg to about 20 mg/kg, about 25 mg/kg to about 50 mg/kg, and about 30 mg/kg to about 60 mg/kg. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

“Effective amount” as used herein refers to an amount of a peptide or peptide conjugate provided herein sufficient to visualize the identified disease or condition (for EGFR-specific peptide conjugates, BE and/or EAC), or to exhibit a detectable therapeutic effect (for GRAIL-1 peptides, e.g., one or more of DNAJA1 binding, inhibiting DNAJA1-Hsp70 chaperone activity, degrading mutant p53 protein, and reducing clonogenic survival of dysplastic BE/EAC cells.) That is, the effect is detected by, for example, an improvement in clinical condition or reduction in symptoms. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

Visualization of binding to BE or EAC cells is by any means known to those of ordinary skill in the art. As discussed herein, visualization is, for example and without limitation, in vitro, in vitro, or in situ visualization.

When the detectable label is a radiolabel, the radiolabel can be detected by nuclear imaging.

When the detectable label is a fluorophore, the fluorophore can be detected by near infrared (NIR) fluorescence imaging.

Methods provided herein can comprise the acquisition of a tissue sample from a patient. The tissue sample can be a tissue or organ of said patient.

### Formulations

Compositions provided herein comprise pharmaceutically acceptable excipients such as carriers, solvents, stabilizers, adjuvants, diluents, etc., depending upon the particular mode of administration and dosage form. The compositions are generally formulated to achieve a physiologically compatible pH, and range from a pH of about 3 to a pH of about 11, about pH 3 to about pH 7, depending on the formulation and route of administration. The pH can be adjusted to a range from about pH 5.0 to about pH 8. The compositions can comprise a therapeutically effective amount of at least one peptide or peptide conjugate as described herein, together with one or more pharmaceutically acceptable excipients. Optionally, the compositions comprises a combination of the compounds described herein, or can include a second active ingredient useful in the treatment or prevention of bacterial growth (for example and without limitation, anti-bacterial or anti-microbial agents), or can include a combination of peptide or peptide conjugates provided herein.

Suitable excipients include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol) wetting or emulsifying agents, pH buffering substances, and the like.

### Other Terminology and Disclosure

As used herein and in the appended claims, the singular forms “a,” “and,” and “the” include plural referents unless the context clearly dictates otherwise. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

When a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of the present disclosure.

All publications mentioned herein are incorporated herein by reference to disclose and describe the methods and/or materials for the purpose for which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible. This disclosure is intended to provide support for all such combinations.

As used herein, “may,” “may comprise,” “may be,” “can,” “can comprise” and “can be” all indicate something envisaged by the inventors that is functional and available as part of the subject matter provided.

### EXAMPLES

While the following examples describe specific embodiments, variations and modifications will occur to those skilled in the art. Accordingly, only such limitations as appear in the claims should be placed on the invention.

### Example 1

### The Efficacy of Pep J±Simvastatin in Degrading Mutant p53 and Abolishing Dysplastic Lesions Present in PDOs Grown In Vitro

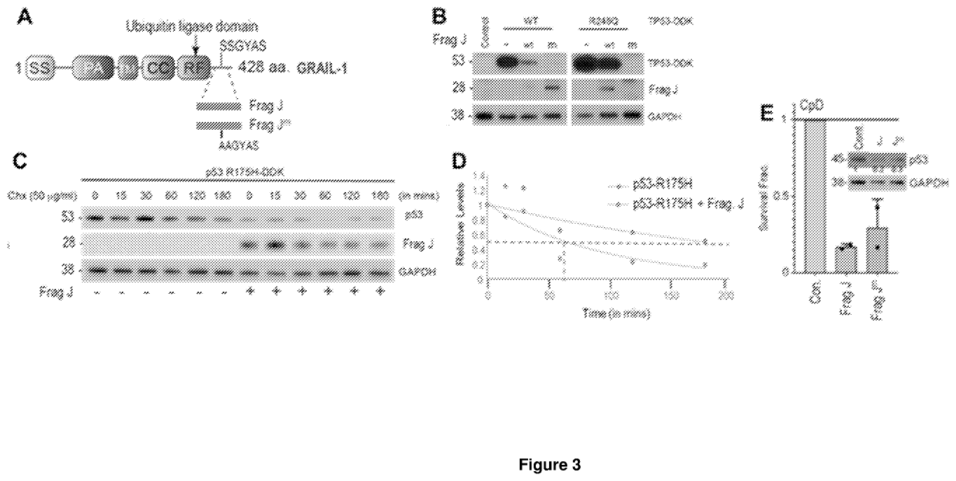

Analyses using a large (>560 samples) tissue bank of untreated BE and EAC samples followed by genomic characterization, gene expression analysis (RNAseq), and protein profiling (tissue microarray, TMA) identified GRAIL Isoform 1 (GRAIL-1) as a critical connector that establishes a molecular interaction with the DNAJA1-Hsp70 co-chaperone complex involved in stabilizing mutant p53. The elucidation of the isoform-specific role of GRAIL-1 and identification of a DNAJA1 binding motif outside the ubiquitin ligase domain of GRAIL-1 found to be essential in stabilizing mutant p53 protein has significant translational potential. Further data was obtained showing that overexpression of a 114 amino acid long GRAIL fragment (Frag J) “locks” the DNAJA1-Hsp70 chaperone complex blocking chaperone activity and causing efficient mutant p53 degradation. Frag J was then narrowed down to a ten amino acid long minimal essential area (315-PMCKCDILKA-324) in GRAIL-1 that is sufficient for DNAJA1 binding. Next, a cell permeable (with HIV TAT-GRKKRRQRRRPQ) peptide (Pep J) was synthesized that phenocopies Frag J activity to cause mutant p53 degradation reducing clonogenic survival of dysplastic BE/EAC cells (FIG. 3). Data was obtained showing that compared to monotherapy, Pep J+simvastatin treatment had a synergistic effect in degrading mutant p53 and reducing clonogenic survival of mutant p53 driven dysplastic BE/EAC cells, with negligible effects on wild p53 containing BE cells. The synergistic potential of Pep J+simvastatin on inducing mutant p53 degradation and effect on survival of dysplastic BE/EAC cells present was then tested in PDOs that better mimic EAC pathophysiology compared to cell lines grown in 2D culture. To carry out the experiments PDOs were established containing cells representing normal squamous, non-dysplastic/dysplastic BE, and EACs that were identified using top cluster-specific genes to confirm cellular identities from single cell RNAseq (scRNAseq) data (performed in an independent study).

### GRAIL-1 Knockdown Reduces Mutant p53 Levels and Clonogenic Survival of EAC Cells

Clonogenic survival assays were performed using techniques described previously [Ray et al., Neoplasia, 13 (7): 570-578 (2011)]. The effects of different siRNAs (e.g., GRAIL Iso1-1, GRAIL Iso1-2, TP53) on clonogenic survival of different BE (CpA, CpD) and EAC (Flo1, OE19, and OE33) cell lines were determined by normalizing the survival fraction of control siRNA-treated group as 1. Similar clonogenic survival assays were performed to determine the effect of Frag J overexpression, and treatments of peptides and simvastatin.

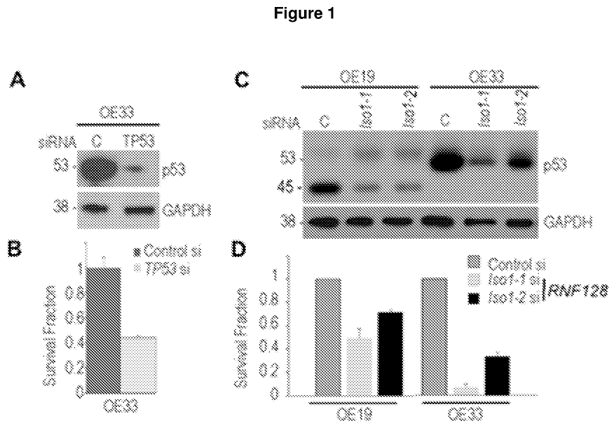

As shown in FIG. 1A and B, loss of mutant p53 in OE33 (a mutant p53 driven EAC cell line), reduced clonogenic survival. As GRAIL-1 is a stabilizer of mutant p53, we have performed GRAIL-1 siRNA (using two independent siRNA) in OE19 and OE33 cells. As shown in FIG. 1C, both the siRNAs reduced mutant p53 levels in both EAC cell lines, which was correlated with reduced clonogenic survival. OE33 cells found to be more sensitive than OE19, suggesting there may be a differential impact based on p53 mutation type (FIG. 1D). These data support GRAIL-1 targeting can promote mutant p53 degradation to kill mutant p53 addicted EAC cells, thus has translational potential.

### a Novel Ubiquitin Ligase-Independent Chaperone-Regulating Function of GRAIL-1

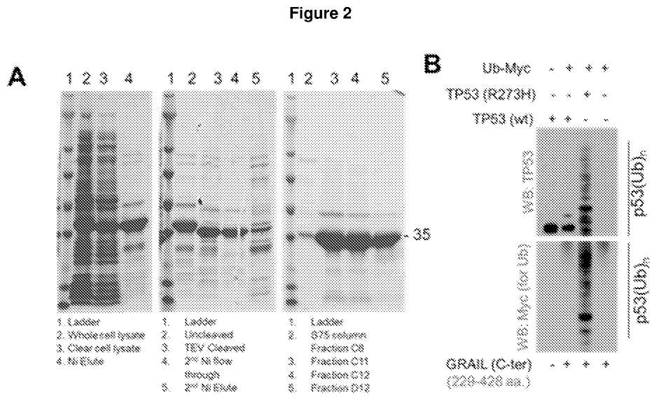

To characterize the ring finger (RF) ubiquitin ligase domain of GRAIL (FIG. 2A), we bacterially expressed and purified His-tagged GRAIL C-terminal end (229-428 amino acids). For easier expression and purification, amino acids 203-228 encoding the transmembrane domain were avoided. Expressed protein was purified using Ni-column (FIG. 2A, left panel). To avoid any complications, His tag was removed using TEV cleavage (FIG. 2A, middle column) and such digested sample was further subjected to size exclusion chromatography using S75 column for further purification. As shown (FIG. 2A, right panel), the purified GRAIL C-terminal fragment ran at a molecular weight of 35 kDa and was used for an in vitro ubiquitination assay.

The in vitro ubiquitination reaction was carried out as described earlier [Shukla et al., Neoplasia, 16 (2): 115-128 (2014)]. Briefly, assays were carried out in 15 μl of reaction volume containing reaction buffer (250 mM Tris-HCl, pH 7.5, 50 mM MgCl₂, 50 PM DTT, 20 mM ATP), 10 μg of Myc-tagged ubiquitin (Cat #U-115), 0.35 μg of UBE1 (Cat #E305), 0.5 μg of UBCH5 (Cat #E2-616) (all from Boston Biochemicals, Cambridge, MA) and bacterially purified 0.5 μg of either the WT or mutant p53 purchased from commercial resources. Human recombinant C-terminal (between amino acids 229-428) protein was purified at the Center for Structural Biology (CSB) core. Such recombinant protein was added as an E3 ubiquitin ligase, and the reaction mixtures were incubated at 37° C. for 2 hours. The reaction was terminated after boiling with 4× gel loading dye. The samples were then resolved and immunoblotted using indicated antibodies. Immunoblotting and immunoprecipitation techniques were performed as described previously (Ray et al. 2011, supra) with a minor modification of buffer, such that excess calcium was added [50 mM HEPES-KOH (pH 7.5), 150 mM NaCl, 1.3 mM CaCl₂), 1 mM dithiothreitol, 10 mM β-glycerophosphate, 1 mM NaF, 0.1 mM sodium orthovanadate, 10% glycerol, 1% NP40 and 1× protease inhibitor cocktail (Sigma, Cat. P8340)].

FIG. 2B shows the result of the assay where we used both wild type and mutant p53 proteins in the presence of recombinant GRAIL (E3), UBCH5C (E2), UBE1 (E1) and Myc-tagged ubiquitin using standardized ubiquitination assay as reported earlier (6). From this study it is evident that a C-terminal RF containing GRAIL fragment is efficient in polyubiquitinating mutant p53 as compared to wild type protein.

### A Deletion Mutant of GRAIL Devoid of RF Domain can Efficiently Degrades Mutant p53.

While analyzing different deletion mutants of the C-terminal fragments of GRAIL, a 114 amino acid long fragment located outside of the RF domain (responsible for ubiquitin ligase activity) was identified (FIG. 3A). This domain encompasses a previously reported phosphodegron for β-TrCP1, required for GRAIL degradation [Ray et al. 2020, supra]. Overexpression of such a fragment (hereby Frag J) or its S to A mutant (Frag J^{m}) resulted in expression of 28 kDa proteins. As shown in FIG. 3B, overexpression of Frag J/J^{m} resulted in loss of both wild type and mutant p53 proteins. Such decrease was due to increased degradation as shown and quantified using a cycloheximide protein half-life study (FIG. 3C). Mutant p53^{R175H} protein half-life in CpD cells were determined by adding cycloheximide (100 μg/ml) 24 hrs after transfection in the presence and absence of Frag J. Cells were harvested at the indicated times post-treatment, then immunoblotting using antibodies to DDK (for p53^{R175H}) and GAPDH as loading control. The approximate p53^{R175H} protein half-life (t_{1/2}) in CpD cells was calculated by plotting relative band density (arbitrary units) and time (hr) in a linear scale.

Mutant p53-R175H with longer half-life (T_{1/2}>200 min) showed reduced stability (T_{1/2}<60 min) following Frag J overexpression (FIG. 3D). When overexpressed in mutant p53 driven CpD cells (a dysplastic BE cell line), Frag J/J^{m} degraded mutant p53 and reduced clonogenic survival (FIG. 3E). Together, this data identified a C-terminal fragment of GRAIL, which is devoid of the ubiquitin ligase domain, yet promoted mutant p53 degradation. The mechanism of such an unexpected observation was explored in the following experiments.

Frag J Interacts with Hsp40 to Modulate Mutant p53 Stability in Ubiquitin Ligase Independent Manner.

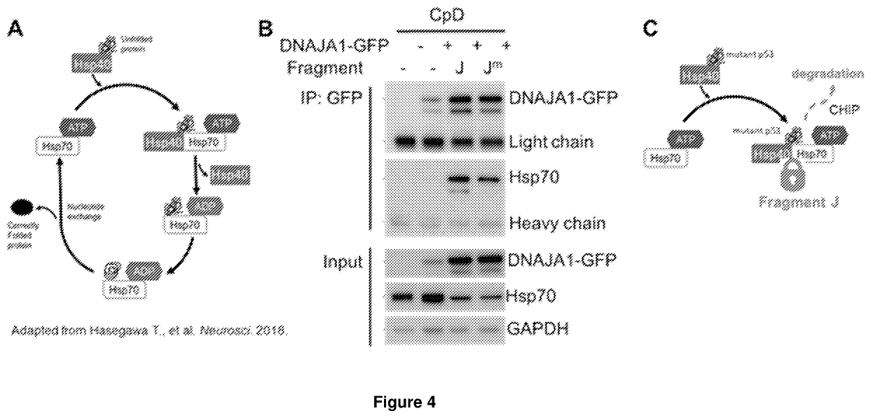

To understand the mechanism of Frag J/J^{m} mediated mutant p53 degradation in a ubiquitin ligase independent manner, Flag-tagged Frag J was over-expressed in the CpD cell line and immunoprecipitation was performed followed by mass spectrometry analysis to identify key interactors. To determine Frag J interactors, CpD cells were transfected using Fugene transfection reagent (Promega) as standardized in the lab. Twenty-four hours post-transfection, cell lysates were subjected to immunoprecipitation using anti-GFP antibody followed by Protein A/G bead pull-down using standardized immunoprecipitation studies. Following three times washing followed by a final wash with ice-cold PBS, beads were submitted to the University of Michigan mass spectrometry core for analysis as described previously [Maine et al., Nature Protocols, 5 (8): 1447-1459 (2010)]. Briefly, upon trypsin digestion, peptides were resolved on a nano-capillary reverse phase column and subjected to high-resolution, linear ion-trap mass spectrometer (LTQ Orbitrap XL, Thermo Fisher). The full MS scan was collected in Orbitrap (resolution 30,000@400 m/z), and data-dependent MS/MS spectra on the nine most intense ions from each full MS scan were acquired. Proteins and peptides were identified by searching the data against Swissprot human protein database, appended with decoy (reverse) sequences, using X!Tandem/Trans-Proteomic Pipeline (TPP) software suite. All proteins identified with a ProteinProphet probability of >0.9 (fdr<1%) were accepted. Spectral matches to identified peptides were manually verified. Multiple different Hsp40 family members were identified, including DNAJA1, as top interactors of Frag J. Hsp40/DNAJA1 co-chaperone activity with Hsp70 is essential for protecting misfolded proteins including mutant p53 to maintain stability. In this process, Hsp40 recognizes misfolded proteins and must transfer them to Hsp70 for refolding. This is ATP-driven dynamic process where following transfer, Hsp40 must dissociate from Hsp70 allowing it to perform chaperoning activity for refolding (FIG. 4A). Since Frag J showed novel interactions with DNAJA1, whether overexpression of Frag J/J^{m} is inhibiting Hsp40-Hsp70 chaperone activity promoting mutant p53 degradation in a ubiquitin ligase independent manner was tested. To confirm, immunoprecipitation studies between Frag J/J^{m} and DNAJA1 were performed. As shown in FIG. 4B, presence of the fragment resulted in a locked DNAJA1-Hsp70 complex formation. Such data supported a model (FIG. 4C) in which following Frag J binding with DNAJA1, it forms a locked complex with Hsp70 inhibiting chaperone activity causing mutant p53 to remain in misfolded state to be degraded by CHIP, a U-box family ubiquitin ligase.

### Frag J is Localized in the Rough ER.

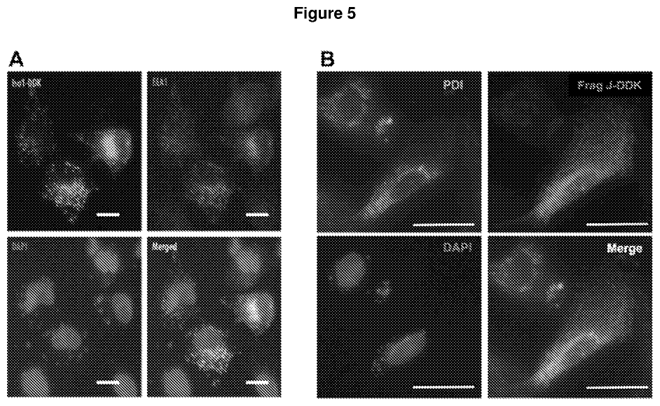

GRAIL is located primarily at the endosome (FIG. 5A) where it colocalizes with an early endosome protein (EEA1). Since Frag J/J^{m} is devoid of transmembrane domain and able to interact with DNAJA1, an experiment was performed to show it localized in the cytosol. As shown in FIG. 5B, Frag J showed fibrillar cytosolic staining, which colocalized with a rough endoplasmic reticulum (ER) marker, PDI. As protein folding often occurs at the ER, Frag J is contemplated to interact with DNAJA1 at the rough ER to inhibit chaperone activity, thus compromising refolding of misfolded mutant p53 and causing its degradation.

### Fragment J Blocks Chaperone Activity.

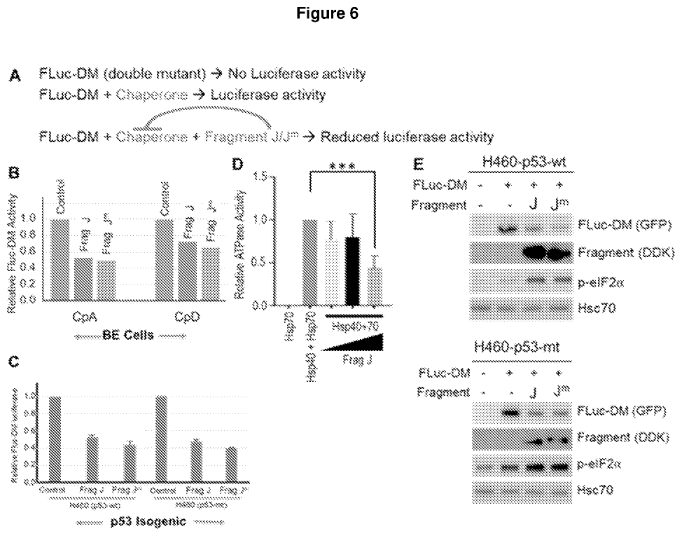

As Frag J/J^{m} overexpression locks DNAJA1-Hsp70 complex, whether such interactions inhibit chaperone activity was tested. A luciferase-based reporter system was developed using a double mutant of luciferase (Fluc-DM), whose activity largely depends on the chaperone activity [Gupta et al., Nature Methods, 8 (10): 879-884 (2011)], and hence can be used as an indirect approach to determine cellular chaperone activity (FIG. 6A). To understand the in vivo effects of the Frag J or different peptides in multiple cell lines including BE/EAC and p53 isogenic lines, cells were first transfected/co-transfected with Fluc-DM±Frag J constructs. Twenty-four hours post-transfection, cells were either subjected to luciferase assay or for peptide treatment studies, cells were treated with 20 μM concentrations of different peptides. After overnight incubation, cell lysates were prepared using lysis buffer provided in the luciferase assay kit in Promega luciferase assay system (cat. E1910) followed by standardized protocol as provided by the manufacturer. Overexpression of Frag J/J^{m} inhibited Fluc-DM luciferase activity, which was independent of presence or absence of p53 mutation status (FIG. 6B, C). Such observation was confirmed by an in vitro chaperone activity assay using recombinant purified Hsp40-Hsp70 complex (FIG. 6D). In Fluc-DM study, loss of Fluc-DM levels following Frag J/J^{m} overexpression (FIG. 6E) was noted as shown using immunoblotting. Such effects were independent of p53 mutation status. Additionally, increased levels of pelF2, a marker for ER stress, were found suggesting that the overexpression of fragment J can cause ER stress, inhibiting chaperone activity to compromise protein folding leading to degradation.

Identification of minimal essential domain of Frag J that interacts with DNAJA1. Frag J-mediated mutant p53 degradation has significant translational potential. Thus, experiments identifying the minimal essential domain in Frag J capable of binding and inhibiting DNAJA1 were performed for translational purposes. First, docking analysis of Frag J with three different PDB structures of DNAJA1 (1HDJ: NMR solution structure of the J-domain of Hsp40; 3AGY: crystal structure of human Hsp40 Hdj1 peptide-binding domain complexed with a C-terminal peptide of Hsp70; and 2QLD: crystal structure of the putative peptide-binding fragment from the human Hsp40 protein Hdj1) was performed using Hex8.0.0. Amino acid sequences A1-10, A71-80 and A101-110 exhibited the lowest docking energy. Amino acid sequences from A1-5 and A101-105 were combined and their binding was assessed using PepSite online tool with PDB code 1HDJ to predict peptide binding probability (p value) on protein surfaces. Alanine scanning was performed to determine amino acids critical for binding. Further amino acid replacements were performed to maximize binding. Promising sequences were analyzed for docking energy using Hex8.0.0. Table 1 summarizes data identifying amino acids 1-10 and 101-114 of Frag J as best possible peptide sequences (negative docking energy) capable of interacting with DNAJA1 using the in silico approaches.

TABLE 1

| | Docking | Sequence |
|---|---|---|
| Frag J Peptides | Energy | Identifier |
| A1-10: PMCKCDILKA | -1289.76 | SEQ ID NO. 10 |
| A11-20: LGIEVDVEDG | -938.01 | SEQ ID NO. 11 |
| A21:30: SVSLQVPVSN | -1170.1 | SEQ ID NO. 12 |
| A31-40: EISNSASSHE | 1013.38 | SEQ ID NO. 13 |
| A41-50: EDNRSETASS | -1066.24 | SEQ ID NO. 14 |
| A51-60: GYASVQGTDE | | SEQ ID NO. 15 |
| A61-70: PPLEEHVQST | -972.02 | SEQ ID NO. 16 |
| A71-80: NESLQLVNHE | -1299.12 | SEQ ID NO. 17 |
| A81-90: ANSVAVDVIP | -1046.86 | SEQ ID NO. 18 |
| A91-100: HVDNPTFEED | | SEQ ID NO. 19 |
| A101-114: ETPNQETAVREIKS | -1606.07 | SEQ ID NO. 20 |

Importantly, although Frag J peptide A71-80 showed binding using Hex 8.0.0, Pepsite 2 prediction failed to confirm binding, hence it was eliminated from further analyses. Next, peptides (Table 2) were designed encompassing the two identified peptide sequences from Table 1, amino acids 1-10 and 101-114 of Frag J, and the peptides were tested for different effects including DNAJA1 binding, inhibition of chaperone activity, mutant p53 degradation and loss of clonogenic survival of mutant p53 driven BE/EAC cells as described below. To conduct both cellular and biochemical assays, two separate sets of peptides were synthesized for each region: one with HIV TAT (GRKKRRQRRRRPQ) for cell permeability and the other without as shown in Table 2.

TABLE 2

| | | | Docking | | Sequence |
|---|---|---|---|---|---|
| Peptide | Peptide Sequence | | energy | P value | Identifier |
| 1 | GRKKRRQRRRPQ | PMCKCDILKA | -1289.76 | 0.1018 | SEQ ID NO. 6 |
| 2 | GRKKRRQRRRPQ | NESLQLVNHE | -1299.12 | 0.2632 | SEQ ID NO. 22 |
| 3 | GRKKRRQRRRPQ | ETPNQETAVR | -1227.2 | 0.07605 | SEQ ID NO. 23 |
| 4 | GRKKRRQRRRPQ | PMCKATPWRE | -1238.74 | 0.02641 | SEQ ID NO. 7 |
| 5 | GRKKRRQRRRPQ | PMCKVPPWRQ | -1239.19 | 0.0114 | SEQ ID NO. 8 |
| 6 | GRKKRRQRRRPQ | PMCKVDPWRQ | -1211.71 | 0.04065 | SEQ ID NO. 24 |
| 7 | GRKKRRQRRRPQ | PMCKVPPWR | -1207.14 | 0.009631 | SEQ ID NO. 9 |
| 8 | GRKKRRQRRRPQ | CKVPPWRQ | -1154.23 | 0.00586 | SEQ ID NO. 25 |
| 9 | GRKKRRQRRRPQ | CKVPPWR | -1059.97 | 0.005208 | SEQ ID NO. 26 |
| 10 | GRKKRRQRRRPQ | KVLQLPPWR | -1228.82 | 0.01615 | SEQ ID NO. 27 |
| 11 | GRKKRRQRRRPQ | KVQLVPPWR | -1224.75 | 0.03225 | SEQ ID NO. 28 |
| 12 | GRKKRRQRRRPQ | LGIEVDVEDG | -938.01 | 0.5149 | SEQ ID NO. 29 |
| 13 | GRKKRRQRRRRPQ | | | | SEQ ID NO. 30 |

HIV TAT [symbol] For cell permeabilization

### Pep 1 and 5 Treatments Reduce Clonogenic Survival of Mutant p53 Driven BE Cells

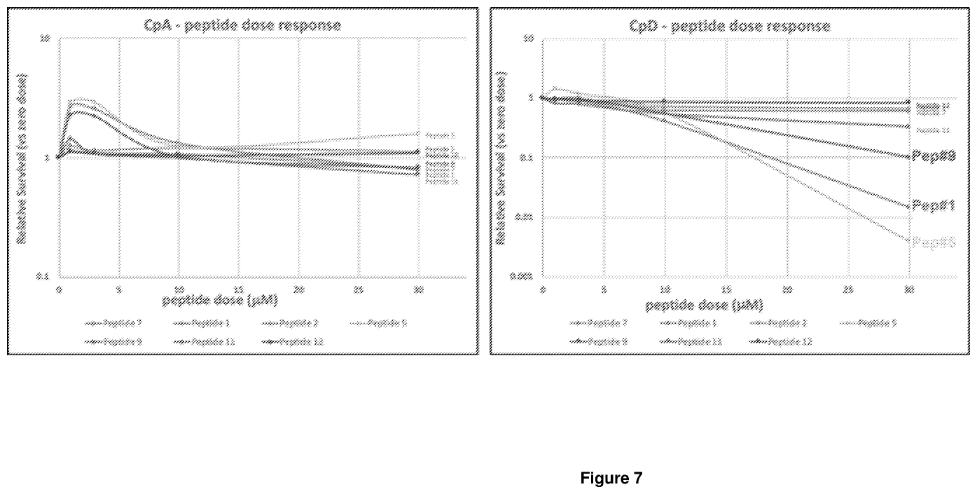

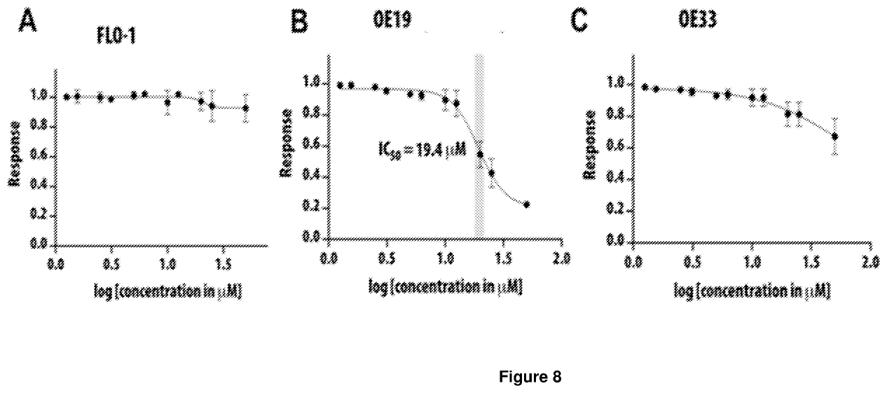

All thirteen peptides (Table 2) were first screened for their efficacy in killing mutant p53 driven dysplastic cells. For this purpose, two BE cell lines were used, CpA (with wild type p53) and CpD (carrying mutant p53). As shown in FIG. 7A, B, four peptides (Pep 1, 5, 9, and 11) were identified with better differential activity in reducing clonogenic survival of CpD cells. Among them, Pep 1 and Pep 5 were the top hits with comparable IC₅₀ values of 11.2±2.3 μM in CpD cells and >30 μM in CpA, indicating the therapeutic potential of these two peptides. In comparison, Pep 11, 12, and 13 were identified as having the least cytotoxic effect on either cell types. When Pep 1 and 5 were tested in EAC cells (Flo1, OE19, and OE33), the EAC cells were found to be relatively more resistant with OE19 found to be the most sensitive (IC₅₀: 19.4 μM) (FIG. 8A-C). The cell viability assay was performed using MTT kit (Roche product #11465007001) and assays were performed according to the manufacturer's protocol. In brief, 3000 cells in 100 μl of complete medium were plated per well in a 96-well plate 24 h prior to peptide treatments. Cells were then either treated with vehicle (dH₂O) or serial dilutions of peptides. Two days following treatments MTT labelling reagent was added, and cells were allowed to form formazan crystals for 2 h. Following, 100 μl of solubilizing agent (10% SDS in 0.01M HCl) was added per well, and plates were incubated at 37° C. overnight. The optical density (OD) of the solubilized formazan was spectrophotometrically quantified using a plate reader at 570 nm with a reference at 650 nm. Data represent the mean (±standard error, SE) performed in quadruplicate, and percent cell viabilities are plotted in semi-logarithmic scale relative to water treated control. In this study, Pep 12 showed no effects on any EAC cell lines.

Pep 1 Better Interacts with DNAJA1 and Inhibits Chaperone Activity

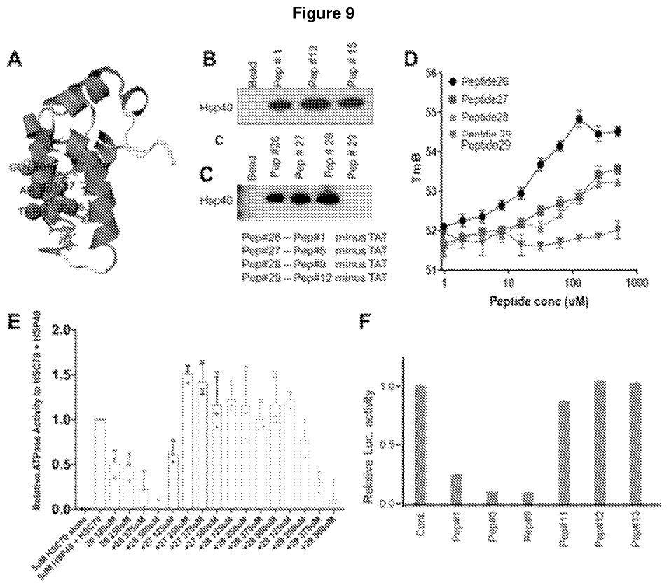

To show direct interaction between peptides and recombinant DNAJA1, multiple assays were performed as shown in FIG. 9. Based on an in silico analysis, Pep 5 showed binding with the J-domain of Hsp40 (FIG. 9A). While performing a peptide pull down assay using NHS activated Sepharose beads, both Pep 1 and 12 showed interactions with the purified DNAJA1. Peptide sequences with or without TAT were procured from Biomatik Corp. Pull down assays were performed as described previously [Jaiswal et al., Antioxid Redox Signal, 36 (1-3): 39-56 (2022)]. Briefly, 1 mg of peptide was dissolved in 1 ml of coupling buffer (0.2 M NaHCO₃, 0.5 M NaCl, pH 8.3). Peptides were conjugated to NHS-activated resin by incubating 100 μL NHS bead slurry with 200 μL peptide solution for 2 hours at room temperature (RT). Unbound sites were blocked with quenching buffer (0.5 M ethanolamine, 500 mM NaCl). Resin peptide conjugates were washed once with low pH (0.1 M acetate, 0.5 M NaCl, pH 5) and high pH buffer (0.1 M Tris HCl, pH 8.0) and equilibrated with lysis buffer 50 mM HEPES, 150 mM NaCl, 0.2% NP-40, protease inhibitor cocktail, pH 7.5. Purified HSP40 protein was procured from Boston Scientific (cat #). Purified protein was diluted in lysis buffer. Approximately 40 nM protein was incubated with peptide coupled to NHS beads and incubated for 2 hours at RT. Unbound proteins were removed by centrifugation and beads were washed with washing buffer (50 mM HEPES, 300 mM NaCl, 0.2% NP-40, pH 7.5). Bound proteins were obtained by boiling beads with 1× Laemmli buffer. Binding was confirmed with Western Blot using anti-His antibody (Abcam, #ab18184). Interesting, Pep 13 that consists of only the HIV TAT sequence also showed interaction suggested stickiness of the TAT sequence (FIG. 9B). To circumvent this, TAT-less Pep 1, 5, 9, and 12 (numbered respectively as Pep 26, 27, 28, and 29) were synthesized and similar peptide pull down studies were performed. As shown in FIG. 9C, Pep 26, 27, and 28 bound effectively to DNAJA1, however, no interaction between Pep 29 and Hsp40 was observed. To confirm, thermal stability assays (TSA) were performed. TSA was carried out using QuantStudio™ 7 Flex Real-Time PCR System. Reactions were carried out in 384 well plate (Applied Biosystems, Cat #4309849) in 10 μl reaction containing 0.2 mg/ml purified protein, 10×SYPRO Orange (Invitrogen, Cat #S6650) dye and peptide (0-500 μM). The plates were covered with optical foil during the reaction in the thermal cycler. Plates were covered with Optical Adhesive Film (Applied Biosystems, 4360954) during the reaction. The Instrument was programmed in the melt curve mode with ROX reagent detection and Fast speed run. The reporter was selected as Rox and none for quencher. Each melt curve was programmed as follows: 25° C. for 15 sec, followed by increase in temperature at the speed of 0.03° C./s up to 95° C. and finally 95° C. for 15 sec. The raw data was exported to Protein Thermal Shift™ Software (Applied Biosystems) for analysis. Melting temperature was calculated using the melt curve. As shown in FIG. 9C, Pep 26 (equivalent to Pep 1+TAT) showed the best result so it was designated the lead peptide. To further test functionality, an in vitro ATPase activity assay was performed using purified Hsp40 and Hsp70 proteins. As shown in FIG. 9D, similar to the in silico binding study results, Pep 26 (TAT-less Pep 1) showed the best efficacy in inhibiting Hsp40-Hsp70 chaperoning activity in a dose-dependent manner. Using the in vivo system of Fluc-DM, inhibition of chaperone activity was similarly observed in the presence of Pep 1, 5, and 9 with minimal inhibition in the presence of Pep 11, 12, and 13 (FIG. 9F). Pep 1 was redesignated “Pep J”.

Lead Peptides are Effective in Degrading Mutant p53 that Correlates with Cytotoxic Effects.

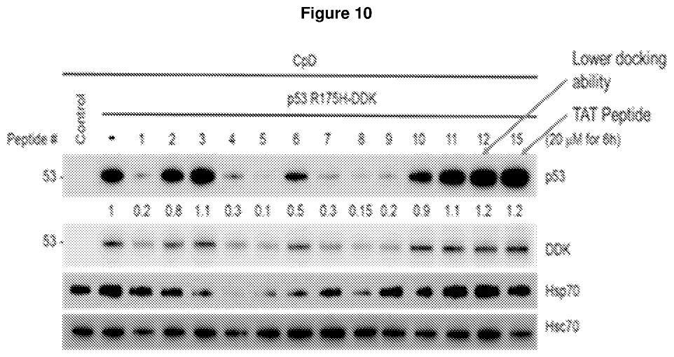

As shown in FIG. 7, Pep 1 and 5 are effective in reducing clonogenic survival. Consequently, the effect of all thirteen peptides on mutant p53 steady state levels was tested. As shown in FIG. 10, Pep 1, 5, and 9 were found to be highly effective in downregulating mutant p53-R175H protein.

### Synthesis of Scrambled Peptides.

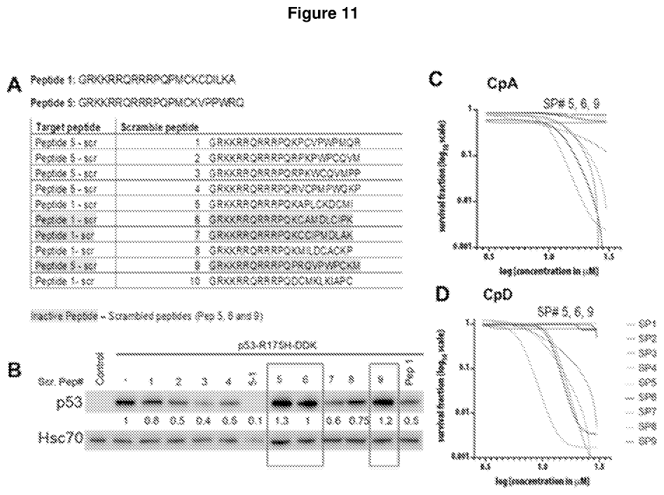

Scrambled forms of Pep 1 and Pep 5 were synthesized for use as negative controls. FIG. 11A shows the sequences of the scrambled peptides. Among them, three peptides (Scr. Pep 5, 6, and 9) showed no effect or even increased levels of mutant p53 (FIG. 11B). Consequently, these peptides showed minimal cytotoxicity towards both CpA and CpD cells (FIG. 11C, D).

### Pep J and Simvastatin Show Synergy in Promoting Mutant p53 Degradation and Reduces Clonogenic Survival of Neoplastic BE Cells.

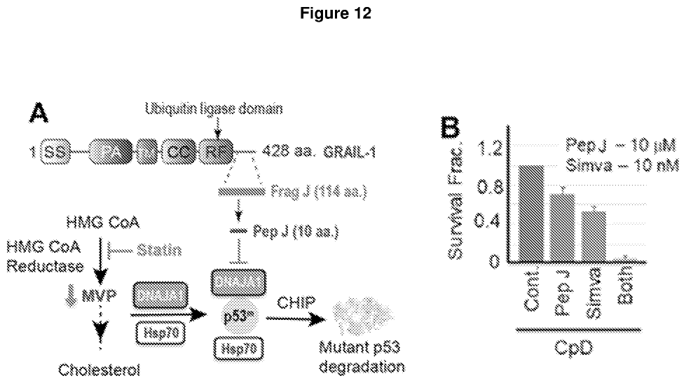

As discussed above, although both Pep J and simvastatin target DNAJA1 to promote mutant p53 degradation, their mechanism of DNAJA1 inhibition is significantly different. Simvastatin reduces MVP synthesis, thus hindering DNAJA1 loading to mutant p53 and compromising its refolding [Parrales, supra], whereas Pep J locks the DNAJA1-Hsp70 complex the dissociation of which is essential for Hsp70 to refold mutant p53 (FIG. 12A). This specific difference provides the rationale for testing Pep J with or without (±) simvastatin on neoplastic BE (CpD) and EAC (OE33) cells.

As shown in FIG. 12B, compared to either alone, dual treatment was highly effective in killing CpD and EAC cells.

### Establishment and Characterization of BE/EAC Organoids

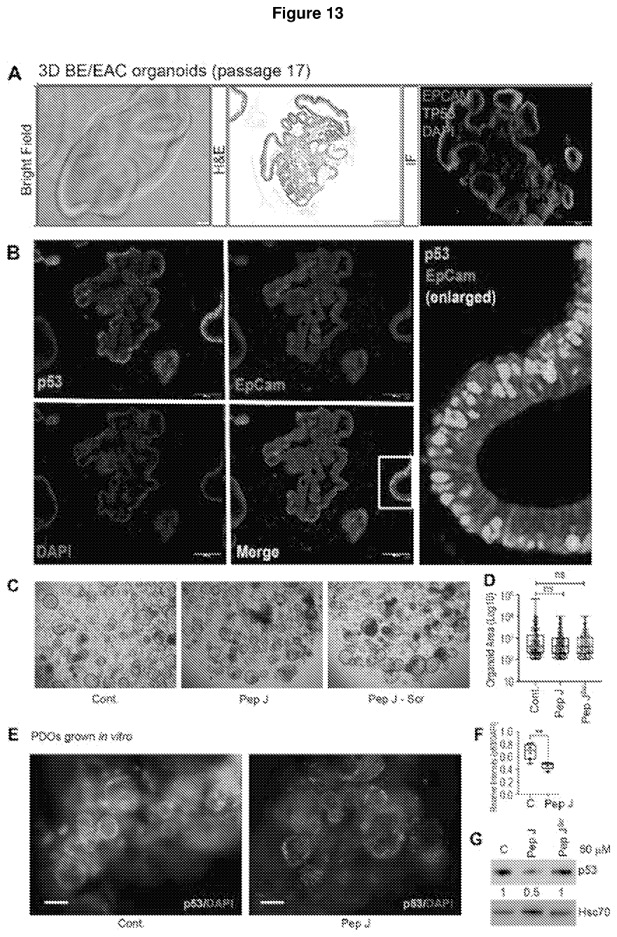

As noted above, due to significant anatomical differences between human and mouse esophagus, prior to the present disclosure there was no transgenic mouse model that effectively represents BE/EAC in vivo. For the experiments described herein, a standardized protocol was developed to establish BE/EAC organoids in which to test the therapeutic effects of DNAJA1 targeting (Pep J±simvastatin) on mutant p53 degradation and PDO growth both in vitro and in vivo. Organoids were derived from tissue biopsies retaining heterogenous cell types and using scRNA seq. The organoids had biomarkers demonstrating normal squamous (NS), non-dysplastic and dysplastic BE esophagus and EAC from three different patients, with matched histology, immunohistochemistry, immunofluorescence (FIG. 13A). Experiments also established the presence of mutant (intense nuclear staining) and wild type (diffuse staining) of p53 (FIG. 13B). The organoids were maintained for over 20 passages.

Tissue specimens were collected under the IRB protocol (HUM00102771) from patients diagnosed with EAC undergoing pre-treatment endoscopy in the Medical Procedures Unit (MPU) at the UM Hospital. Such pretreatment biopsies were collected according to approved protocols. Once a patient is identified, the tissue donor signs an informed consent document that allows access to relevant coded clinical information and pathological annotation of samples. The consent document allows for genetic analysis (both DNA and RNA sequencing) and allows for sharing of de-identified tissue and data with other investigators. Thus, all of the relevant clinical information, mutational analysis and histopathology were available. Patient samples were collected (up to twelve 3×3 mm biopsy specimens per patient), and immediately placed in a tube containing ice cold Advanced DMEM and placed on ice for transport to the lab for processing and organoid development. Samples were coded and tracked using a password protected online database. Within this database, the samples were linked to downstream organoid/cell culture lines generated, cryopreserved and frozen specimens, formalin fixed, and paraffin embedded (FFPE) tissue blocks or sections, etc. All protocols are in compliant with good clinical practice.

Following tissue arrival at the laboratory, half of the tissue biopsies were pooled and dissociated as whole live cells, to be used for scRNAseq and organoid generation, and the other half of biopsies were formalin fixation in order to prepare FFPE tissue blocks and sections for spatial localization analysis. For whole cell dissociation, a Papain based enzymatic dissociation protocol was used to allow generation of robust scRNA-seq data along with organoids from the same single cell suspension. One of the advantages of the Papain based method is that the enzyme is active at a wide range of temperatures, and although activity is lower at colder temperatures compared to warmer temperatures, it allows a very gentle digestion at 10° C. This is advantageous because colder temperatures have been shown to significantly slow down mammalian cellular machinery, preserving the transcriptome and improving scRNAseq data quality. This dissociation method resulted in tissue viability greater than 90%, and the Papain method, coupled with single cell encapsulation on the 10× Chromium platform and sequencing on the HiSeq4000, yielded high quality scRNAseq data sets capturing 7,000-10,000 cells per sample with a sequencing depth of 50,000 reads/cell to obtain a median of ˜2500 genes/cell. scRNA-seq data generated demonstrated the presence of normal squamous (NS), Non-dysplastic Barrett's Esophagus (NDBE), Barrett's Esophagus and EAC from patients, with matched histology, immunohistochemistry, immunofluorescence and scRNA-seq data showing cellular heterogeneity.

### Simvastatin and Pep J Show Synergy in Promoting Mutant p53 Degradation and Reduced Clonogenic Survival of Dysplastic BE Cells

The effect of Pep J and simvastatin treatment either alone or in combination on wild type and mutant p53 was tested in BE/EAC cells present in ten established PDOs grown in vitro. Scrambled Pep J was used as a negative control. As shown in FIG. 13, PDOs were plated as droplets and allowed to grow for 2 days before treatment as follows: DMSO, Pep J, Pep J scrambled (Pep J^{Scr}), simvastatin, and combinations of Pep J/J^{Scr}+simvastatin. Different concentrations of Pep J (10-200 μM) and simvastatin (50-1000 nM) were tested as well as different treatment time periods (24-96 hours). Following completion of treatments, PDOs were either formalin fixed, paraffin embedded, and sectioned to study cellular alterations using immunohistochemistry (IHC) and immunofluorescence (IF) for mutant p53 as shown in FIG. 13A-F. Immunofluorescence staining using different antibodies were performed as described previously (Ray et al. 2011, supra) and fluorescent images were acquired using a DS-Fi1 (Nikon, Melville, NY) camera fitted on an Olympus 1X-71 microscope.

Organoid lysates were prepared for immunoblotting similar to FIG. 13G.

While significant changes in the size of the organoids were not observed within 72 hours of treatment, a 50% reduction in mutant p53 levels in the presence of Pep J was observed by immunoblotting of whole PDO lysis (FIG. 13G). A similar reduction was noted by immunofluorescence staining of FFPE fixed and sectioned organoids (FIG. 13E-F).

### Example 2

### Correlation Between Pep J+Statin Induced Mutant p53 Loss and Reduction in Cell Surface EGFR Expression (QRH*-Cy5)

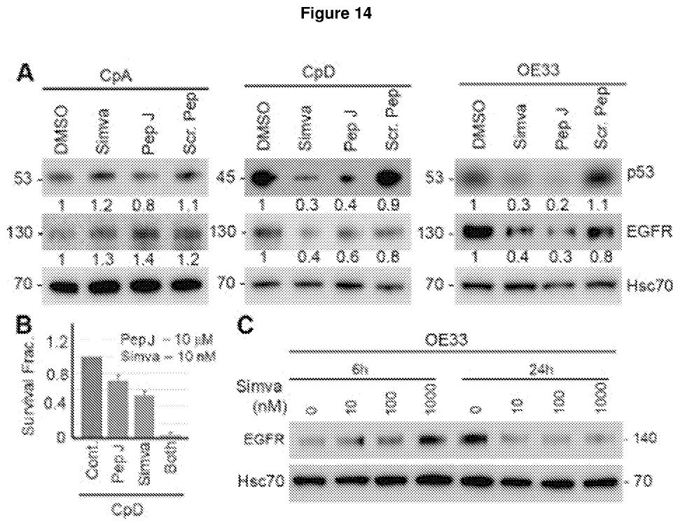

As discussed above, the QRH*-Cy5 peptide can be used to detect cell surface EGFR expression in dysplastic BE/EAC lesions by endoscopy. A new finding, described below, made while testing the effects of Pep J and simvastatin, was a correlation of the loss of mutant p53 and EGFR (FIG. 14A, C) with the killing of dysplastic BE/EAC cells. In view of that finding, the present disclosure contemplates using QRH*-Cy5 peptide to monitor therapeutic responses to BC/EAC treatment.

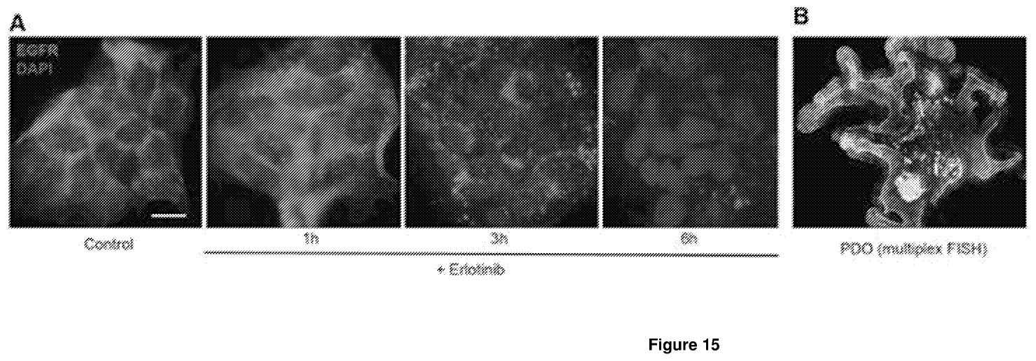

For additional experiments, control and treated (Pep J, simvastatin, combination-different dose and time), FFPE fixed and sectioned PDOs from Example 1 can be used. PDO sections are stained for multiplexed IHC of multiplex FISH using p53, EGFR, cleaved PARP (cell death), Ki67 (proliferation) markers to establish correlation of expression between mutant p53 and EGFR loss. Cell death and proliferation markers are used to correlate mutant p53/EGFR loss with treatment outcome. FIG. 15A shows the gradual loss over time of EGFR expression following Hcc827 cells following drug (erlotinib) treatment. Fixation methods were optimized for fluorescent immunolabeling on the PDOs, alongside clearing methods that enhance high resolution imaging showing multiplexed fluorescence in situ hybridization (FISH) capable of detecting multiple markers (FIG. 15B).

Although multiplexed IHC and multiplexed FISH provide mutant p53/EGFR expression on a cell by cell basis, it is semi-quantitative. To quantify EGFR levels in control and treated (Pep J, Pep J^{Scr}, simvastatin, and combination) PDOs, whole organoid lysates are subjected to immunoblotting using EGFR and p53 antibodies, and GAPDH, β-actin, and Hsc70 expression are used as loading controls.

To confirm the importance of DNAJA1 signaling as a hub to facilitate mutant p53- and EGFR-mediated dysplastic BE to EAC progression, Lentivirus CRISPR mediated gene silencing can be used as an alternate approach to study the effect of knockdown on heterogenous cell types present in organoids.

### Example 3

### Monitoring Efficacy of Pep J±Simvastatin Treatment by In Vivo Endoscopic Imaging of EGFR (QRH*-Cy5) of Implanted PDOs in the Colon of Immunocompromised Mice

PDOs were implanted in the colon of immunocompromised mice as an in vivo model system of human EAC to evaluate the therapeutic efficacy of Pep J+simvastatin. Cultures were harvested from Matrigel in cold DPBS, triturated 30× with a 1 mL pipette tip, and centrifuged at 300 g for 3 min at 4° C. The organoids pellet was resuspended in 10 mL of cold DPBS and mechanically disassociated with the gentle MACS Octo Dissociator (130-096-427, Miltenyi Biotec) using the programs h_Tumor_01.01 followed by m_Lung-01.01. The colonoid fragments were further dissociated by 20× pipetting with a 1 mL pipette tip. Large fragments were removed over a 100 μm BSA-coated cell strainer (#DL 352360, Corning). Slow centrifugation at 100 g was performed to reduce the single cell content. The cell aggregates were resuspended in cold DPBS supplemented with 5% Matrigel and 10 UM Y27632. 1.8×10⁶ cell aggregates in 200 μL were transplanted per mouse as described previously. The implants were monitored over time using a rigid wide-field endoscope and a flexible fiber-coupled endomicroscope to localize the PDO and visualize EGFR expression with sub-cellular resolution.

### Simvastatin Inhibited EAC Tumor Growth in Nude Mice by Promoting Mutant p53 Degradation

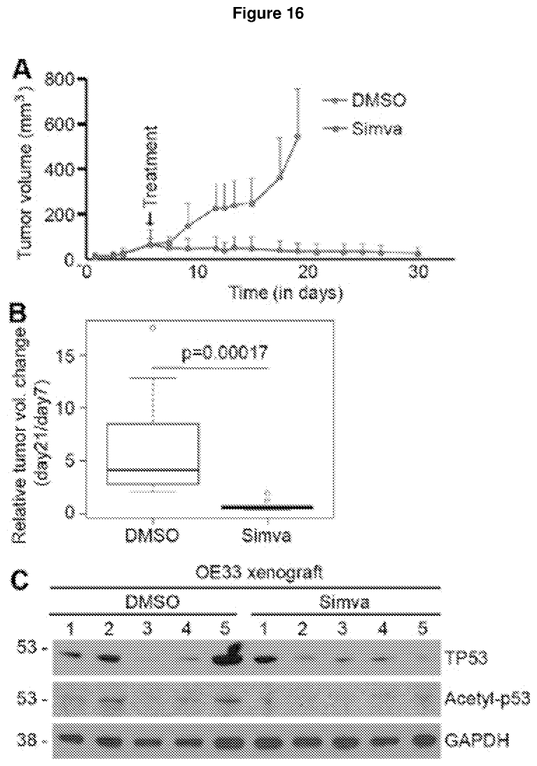

As shown in FIGS. 16A,B, treatment with simvastatin (20 mg/kg per day for 5 days) of mutant p53-driven OE33 tumors in nude mice inhibited growth compared to DMSO control. Such inhibition of tumor growth was correlated with the loss of total and active (acetylated at K382) mutant p53 in simvastatin treated group (FIG. 16C).

### Establishment of Organoid Transplanted Model

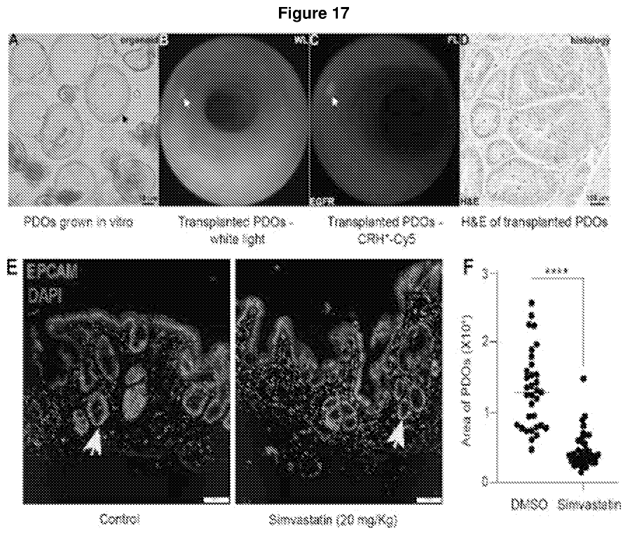

SCID mice (005557 NOD Cg-Prkdc<scid>Il2rg<tm1Wjl>SzJ) were treated with 2.5% dextran sulfate sodium (DSS) for 5 days to induce acute inflammation resulting in focal epithelial damage in the mucosa to allow the organoids to implant and grow. Organoids grown in vitro (FIG. 17A) were implanted on day 7 post-DSS treatment. For implantation, in vitro grown PDOs were disrupted into smaller aggregates in 200 μL volume and were implanted intrarectally in anesthetized mice. The rectum was closed immediately using tissue glue. After several weeks to establish viability, the implanted PDOs were imaged using a rigid wide-field endoscope. The fluorescently-labeled peptide QRH*-Cy5 was administered intravenously at a dose of 300 μM in 200 μL of PBS. As shown in FIG. 17B,C, organoid viability was confirmed after 3 weeks of growth using routine histology (H&E) of resected specimens of mouse colon (FIG. 17D).

### Simvastatin Treatment Reduces Organoid Size Grown In Vivo

As a proof of principal to study the in vivo effects of simvastatin in an implanted PDO model, growth was monitored using fluorescence images collected with intravenous administration of QRH*-Cy5˜3 weeks post implantation. Mice were grouped into two categories and injected intraperitoneally either with DMSO (control) or simvastatin (20 mg/kg single dose). Two days post treatment, mice were euthanized, and the colon was resected, and processed for histology. As shown in FIG. 17E and quantified in FIG. 17F, a reduction in the organoid size in the simvastatin treated group was evident following EpCam (an epithelial marker) staining.

### In Vivo Confocal Imaging

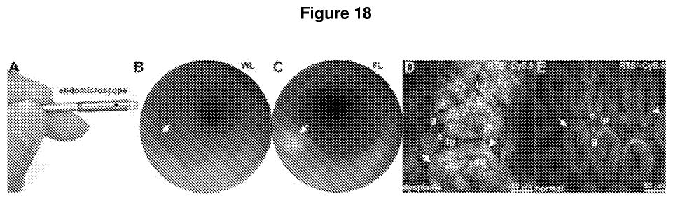

A side-view confocal endomicroscope was used to collect NIR fluorescence images from implanted PDOs with subcellular resolution in vivo to further demonstrate EGFR expression as an imaging biomarker for therapeutic efficacy of Pep J+simvastatin (FIG. 18A). This instrument has a diameter of 4.2 mm, which is small enough to perform repetitive in vivo imaging in mouse colon. The focusing optics provide a spatial resolution of 1.2 μm. A rigid wide-field endoscope was used first to collect white light (WL) and NIR fluorescence (FL) images to identify the approximate location of the implanted PDOs. Data with colonic PDOs is shown in FIG. 18B,C. Landmarks defined by the clockwise location of and distance from the anus to the distal tip of the endoscope were used to approximately locate the implanted PDOs for collection of confocal images. Tumor size and volume were estimated using a calibrated gauge passed through the instrument channel of the rigid endoscope. Preliminary data using a peptide specific for claudin-1 demonstrates the ability of this novel instrument to visualize individual cells in vivo. Differences in the spatial pattern of target expression between dysplasia and surrounding normal mucosa for colonic PDOs can be seen (FIG. 18D,E).

### Ex Vivo Validation

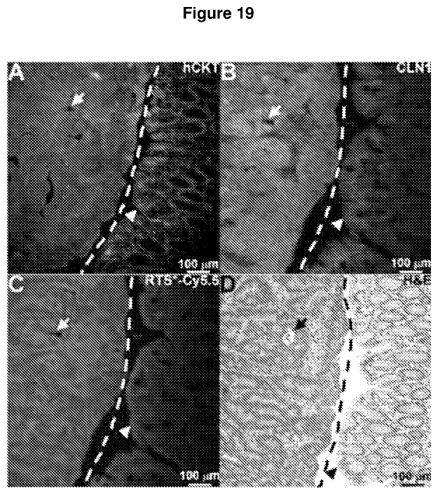

After completion of in vivo imaging, the results were validated using IF. A monoclonal antibody was used to stain the tissue specimen after resection that confirmed the presence of human cytokeratin (hCKT) in the PDO (arrow) adjacent to normal mouse colonic mucosa (arrowhead) (FIG. 19A). Anti-claudin-1 and RTS*-Cy5.5 staining showed overexpression of claudin-1 from the PDO by comparison with mouse colon (FIG. 19B, C). Routine histology (H&E) confirms the presence of human colonic dysplasia adjacent to normal mouse colonic mucosa, (FIG. 19D).

### Experiments in the Mouse Model to Demonstrate the Efficacy of Pep J+Simvastatin

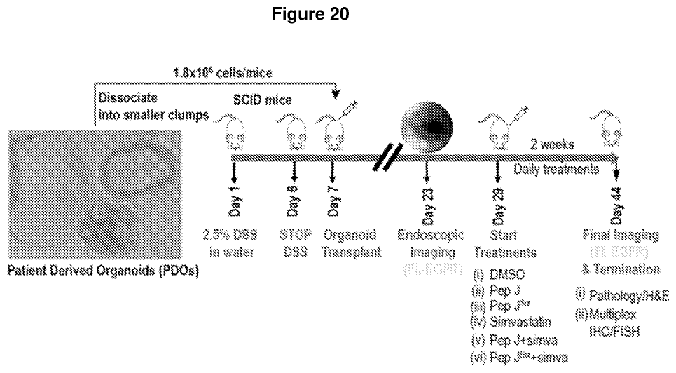

FIG. 20 shows the design of experiments demonstrating the efficacy of Pep J+simvastatin. SCID mice are treated with DSS for the first 5 days followed by PDO transplantation on day 7. During implantation, mice are anesthetized and maintained in that state via a nose cone with inhaled isoflurane mixed with oxygen at concentrations of 2%-4% at a flow rate of 0.5 L/min. Approximately ˜1.8×106 cell aggregates in 200 μL suspension are injected intrarectally using feeding gavage. The rectum is sealed immediately using tissue glue (SC361931, Santa Cruz). PDO implantation and growth are monitored by white light endoscopy. Three weeks post implantation, the following experiments are performed to evaluate the therapeutic potential of Pep J/simvastatin either as a monotherapy or in combination:

Briefly, following confirmation of organoid growth, mice are randomly subdivided into 6 groups having 12 mice in each group: (i) DMSO, (ii) Pep J (100 mg/Kg per day for 2 weeks), (iii) Pep JScr (100 mg/Kg per day for 2 weeks) (iv) simvastatin (20 mg/Kg per day for 2 weeks), (v) Pep J+simvastatin, and (vi) Pep JScr+simvastatin. At the end of the 2 weeks of treatment mice are euthanized and colon is harvested, FFPE fixed and sectioned for H&E, IHC/IF. Multiplexed IHC/FISH is performed for p53, EGFR, cleaved PARP, and Ki67 to evaluate therapy response from different subgroups. A TUNEL assay is separately performed to quantitate cell death. Four different PDOs from Example 1 can be used (three with mutant p53 and one with wild type p53). The size of the transplanted organoids is compared between control and treated groups. Maximum inhibition of mutant p53 driven organoid growth in Pep J+simvastatin treated group is expected compared to monotherapy and DMSO control. In addition, a reduction in mutant p53 and EGFR levels in Pep J+simvastatin treated group is expected with increased positive staining of cleaved PARP, TUNEL positive cells, and decreased numbers of Ki67 positive cells.

Mice are imaged twice using endoscopy after applying EGFR binding peptide (QRH*-Cy5) to monitor EGFR expression at the initiation and termination of treatments as indicated in FIG. 20. A decrease in fluorescence signal post treatment will indicate a response to therapy. Based on in vitro data, a >50% loss in EGFR fluorescence intensity is expected in Pep J+simvastatin treated group with PDOs carrying mutant p53.

As noted, the experiments are carried out using four different PDOs (three with mutant and one with wild type p53). There are 6 different treatment groups having 12 SCID mice per group (sample size calculations designed to give 85% power and an alpha value of 0.05). Normality is assessed using the Kolmogorov-Smirnov and the Shapiro-Wilks test. For variables following a normal distribution, the statistical significance is estimated using the student's t-test (with Welch's correction in case of heterogeneity of variance).

### Summary Relating to Examples 1-3

In summary, the results described in the Examples support and the present disclosure contemplates, DNAJA1 targeting as a novel approach to kill mutant p53 driven dysplastic BE/EAC cells using two mechanistically distinct molecularly targeted agents, Peg J and simvastin. In addition, the present disclosure contemplates the use of an EGRF biomarker peptide to identify dysplastic BE/EAC lesions in vivo and to track the therapeutic response to the DNAJA1-targeting treatment. Moreover, as the current standard of care (chemoradiotherapy) so far provides a survival advantage to only to a minority (<20%) of EAC patients, treatment with PepJ+simvastin is contemplated as a part of neoadjuvant therapy to improve radiotherapy outcome.

### Example 4

### Radiosensitization of Esophageal Adenocarcinoma Cells

OE33, OE 19 and Flo1 esophageal adenocarcinoma (EAC) cells were plated and after overnight incubation treated with 20 μM of Pep J for an additional 24 hours. Following, cells were irradiated with different doses (0, 2, 4, 6, 8 Gy) of radiation. Cells were then incubated for 24 hours prior to plating at a clonal density to perform clonogenic assays. Survival data from a representative experiment are shown in FIG. 1A-C.

In FIG. 1D, radiation enhancement ratios (ER) for OE33, OE19 and Flo1 cells are plotted from three independent experiments and plotted as mean±SE.

Next, OE33, OE 19 and Flo1 EAC cells were treated with 20 μM of Pep J (Peptide 1) or Peptide 5, or with negative control Peptide 12 (Scrambed Peptide 5) or Peptide 13. Twenty-four hours post-treatment cells were harvested, and lysates were subjected to immunoblotting for p53 protein levels. Hsc70 served as a loading control. Data are shown in FIG. 1E.

The experiments show that Pep J radiosensitized EAC cells by promoting misfolded mutant p53 degradation.

### Example 5

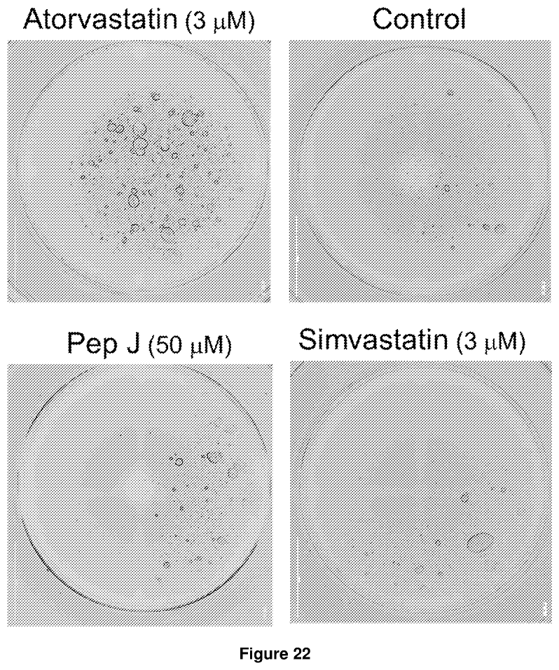

Patient-derived organoids (PDOs) cultured in vitro were treated with simvastatin, atorvastatin, and Pep J for a course of 7 days. FIG. 22 shows Pep J killed PDOs in vitro.

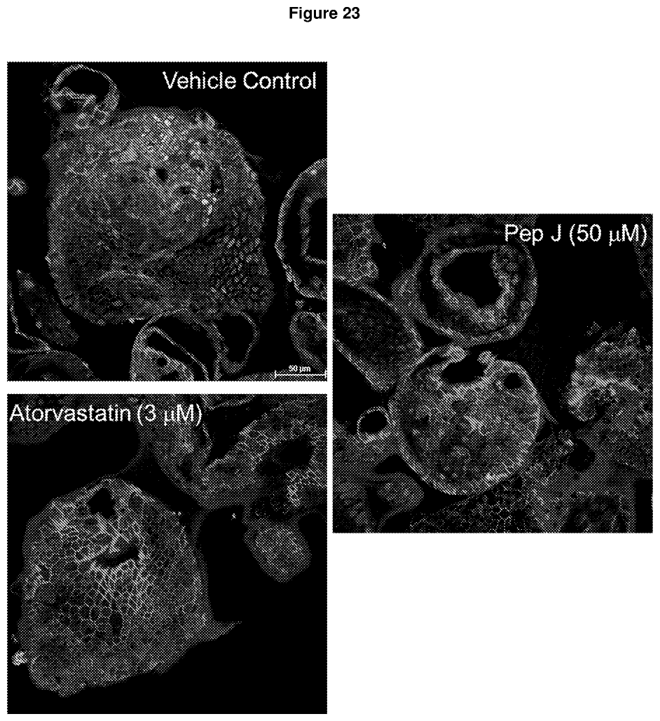

The effect of statin and Pep J on mutant p53 levels in PDOs grown in vitro was also determined. The data shown in FIG. 23 suggests the loss of misfolded mutant p53 within 24 h post treatment.

### Example 6

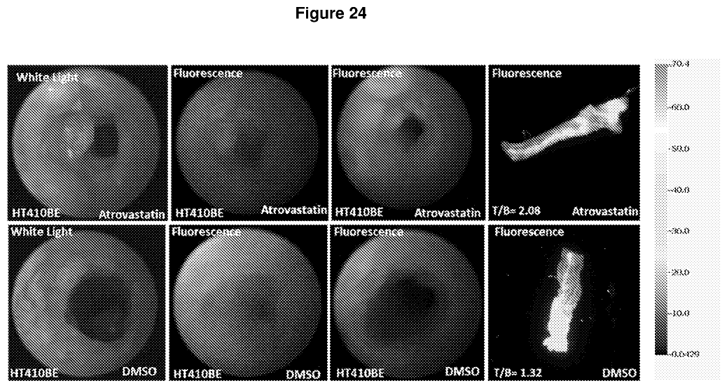

Barrett's esophagus (BE) organoids were implanted on DSS treated mouse colon. On day 21 post implantation (p.i.), the mouse was treated with DMSO/atorvastatin. On day 23 p.i., colon was harvested and fixed in paraffin. Fluorescence endoscopic images were collected pre and post treatment using KCC.Cy5.5 peptide. Post harvest, colon was imaged with PEARL triology at 700 nm. Images are shown in FIG. 24.

### Example 7

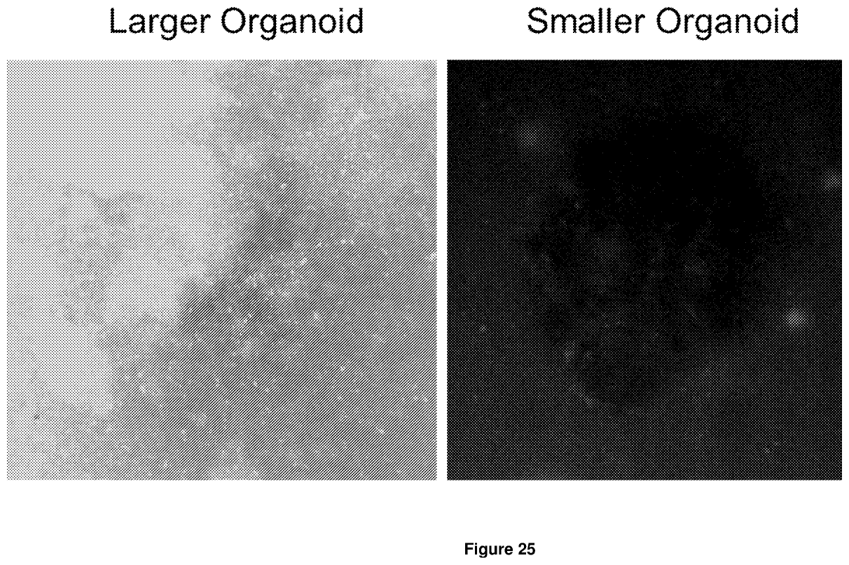

Confocal Z-stack images in FIG. 25 showed entry of Pep J into PDOs.

We claim:

1. A GRAIL-1 peptide comprising amino acids PMCKCDILKA (amino acids 315-324 of SEQ ID NO: 1), or a peptide analog thereof that specifically binds to DNAJA1.

2. A GRAIL-1 peptide of claim 1 comprising the amino acids PMCKCDILKA (SEQ ID NO: 2), or a peptide analog thereof comprising the amino acids PMCKATPWRE (SEQ ID NO: 3), PMCKVPPWRQ (SEQ ID NO: 4) or PMCKVPPWR (SEQ ID NO: 5).

3. A GRAIL-1 peptide conjugate comprising a GRAIL-1 peptide or peptide analog of claim 1 or 2.

4. The GRAIL-1 peptide conjugate of claim 3 further comprising HIV TAT amino acids GRKKRRQRRRPQ.

5. The GRAIL-1 peptide conjugate of claim 4 comprising the amino acids GRKKRRQRRRPQPMCKCDILKA (SEQ ID NO: 6), GRKKRRQRRRPQPMCKATPWRE (SEQ ID NO: 7), GRKKRRQRRRPQPMCKVPPWRQ (SEQ ID NO: 8) or GRKKRRQRRRPQPMCKVPPWR (SEQ ID NO: 9).

6. The GRAIL-1 peptide, peptide analog or GRAIL-1 peptide conjugate of any preceding claim, wherein the GRAIL-1 peptide, peptide analog or GRAIL-1 peptide conjugate exhibits one or more of: specifically binding DNAJA1, inhibiting DNAJA1-Hsp70 chaperone activity, degrading mutant p53 protein, and reducing clonogenic survival of dysplastic BE/EAC cells.

7. A composition comprising a pharmaceutically acceptable diluent and a GRAIL-1 peptide, peptide analog or GRAIL-1 peptide conjugate of any preceding claim.

8. A method for degrading mutant p53 in a cell comprising administering to a cell the composition of claim 7.

9. A method for treating a mutant p53-related disease condition in a patient comprising administering to the patient, the composition of claim 7.

10. The method of claim 9 wherein the mutant p53-related disease condition is Barrett's esophagus, dysplasia, esophageal cancer, oral cancer, nasopharyngeal cancer, laryngeal cancer, head and neck cancer, lung cancer, ovarian cancer, pancreatic cancer or colorectoral cancer.

11. A method for treating for treating Barrett's esophagus (BE) or esophageal adenocarcinoma (EAC) in a patient comprising administering to the patient, the composition of claim 7.

12. A method for monitoring the status of Barrett's esophagus (BE) or esophageal adenocarcinoma (EAC) in a patient treated with a GRAIL-1 peptide composition of claim 7, wherein the method comprises administering an EGFR-specific peptide conjugate comprising a detectable label to the colon of the patient to detect EGFR expressed on the surface of BE and EAC cells in the colon.

13. The method of claim 12 wherein the detectable label is detectable by optical, photoacoustic, ultrasound, positron emission tomography or magnetic resonance imaging.

14. The conjugate of claim 13 wherein the label detectable by optical imaging is fluorescein isothiocyanate (FITC), Cy5, Cy5.5 or IRdye800.

15. The method of claim 12 wherein the EGFR-specific peptide conjugate is QRH*-Cy5.

16. The method of claim 12 wherein the EGFR-specific peptide conjugate is QRH*-KSR*-IRDye800.


SEQUENCE LISTING (44 sequences)

SEQ ID NO: 1 - protein, 428 aa, Homo sapiens
MGPPPGAGVSCRGGCGFSRLLAWCFLLALSPQAPGSRGAEAVWTAYLNVSWRVPHTGVNRTVWELSEEGVYGQDSPLEPVAGVLVPPDGPGALNACNPHTNFTVPTVWGSTVQVSWLALIQRGGGCTFADKIHLAYERGASGAVIFNFPGTRNEVIPMSHPGAVDIVAIMIGNLKGTKILQSIQRGIQVTMVIEVGKKHGPWVNHYSIFFVSVSFFIITAATVGYFIFYSARRLRNARAQSRKQRQLKADAKKAIGRLQLRTLKQGDKEIGPDGDSCAVCIELYKPNDLVRILTCNHIFHKTCVDPWLLEHRTCPMCKCDILKALGIEVDVEDGSVSLQVPVSNEISNSASSHEEDNRSETASSGYASVQGTDEPPLEEHVQSTNESLQLVNHEANSVAVDVIPHVDNPTFEEDETPNQETAVREIKS

SEQ ID NO: 2 - protein, 10 aa, synthetic construct
PMCKCDILKA

SEQ ID NO: 3 - protein, 10 aa, synthetic construct
PMCKATPWRE

SEQ ID NO: 4 - protein, 10 aa, synthetic construct
PMCKVPPWRQ

SEQ ID NO: 5 - protein, 9 aa, synthetic construct
PMCKVPPWR

SEQ ID NO: 6 - protein, 22 aa, synthetic construct
GRKKRRQRRRPQPMCKCDILKA

SEQ ID NO: 7 - protein, 22 aa, synthetic construct
GRKKRRQRRRPQPMCKATPWRE

SEQ ID NO: 8 - protein, 22 aa, synthetic construct
GRKKRRQRRRPQPMCKVPPWRQ

SEQ ID NO: 9 - protein, 21 aa, synthetic construct
GRKKRRQRRRPQPMCKVPPWR

SEQ ID NO: 10 - protein, 10 aa, synthetic construct
PMCKCDILKA

SEQ ID NO: 11 - protein, 10 aa, synthetic construct
LGIEVDVEDG

SEQ ID NO: 12 - protein, 10 aa, synthetic construct
SVSLQVPVSN

SEQ ID NO: 13 - protein, 10 aa, synthetic construct
EISNSASSHE

SEQ ID NO: 14 - protein, 10 aa, synthetic construct
EDNRSETASS

SEQ ID NO: 15 - protein, 10 aa, synthetic construct
GYASVQGTDE

SEQ ID NO: 16 - protein, 10 aa, synthetic construct
PPLEEHVQST

SEQ ID NO: 17 - protein, 10 aa, synthetic construct
NESLQLVNHE

SEQ ID NO: 18 - protein, 10 aa, synthetic construct
ANSVAVDVIP

SEQ ID NO: 19 - protein, 10 aa, synthetic construct
HDVNPTFEED

SEQ ID NO: 20 - protein, 14 aa, synthetic construct
ETPNQETAVREIKS

SEQ ID NO: 21 - protein, 13 aa, synthetic construct
GRKKRRQRRRRPQ

SEQ ID NO: 22 - protein, 22 aa, synthetic construct
GRKKRRQRRRPQNESLQLVNHE

SEQ ID NO: 23 - protein, 22 aa, synthetic construct
GRKKRRQRRRPQETPNQETAVR

SEQ ID NO: 24 - protein, 22 aa, synthetic construct
GRKKRRQRRRPQPMCKVDPWRQ

SEQ ID NO: 25 - protein, 20 aa, synthetic construct
GRKKRRQRRRPQCKVPPWRQ

SEQ ID NO: 26 - protein, 19 aa, synthetic construct
GRKKRRQRRRPQCKVPPWR

SEQ ID NO: 27 - protein, 21 aa, synthetic construct
GRKKRRQRRRPQKVLQLPPWR

SEQ ID NO: 28 - protein, 21 aa, synthetic construct
GRKKRRQRRRPQKVQLLPPWR

SEQ ID NO: 29 - protein, 22 aa, synthetic construct
GRKKRRQRRRPQLGIEVDVEDG

SEQ ID NO: 30 - protein, 12 aa, synthetic construct
GRKKRRQRRRPQ

SEQ ID NO: 31 - protein, 22 aa, synthetic construct
GRKKRRQRRRPQKPCVPWPMQR

SEQ ID NO: 32 - protein, 22 aa, synthetic construct
GRKKRRQRRRPQRPKPWPCQVM

SEQ ID NO: 33 - protein, 22 aa, synthetic construct
GRKKRRQRRRPQRPKWCQVMPP

SEQ ID NO: 34 - protein, 22 aa, synthetic construct
GRKKRRQRRRPQRVCPMPWQKP

SEQ ID NO: 35 - protein, 22 aa, synthetic construct
GRKKRRQRRRPQKAPLCKDCMI

SEQ ID NO: 36 - protein, 22 aa, synthetic construct
GRKKRRQRRRPQKCAMDLCIPK

SEQ ID NO: 37 - protein, 22 aa, synthetic construct
GRKKRRQRRRPQKCCIPMDLAK

SEQ ID NO: 38 - protein, 22 aa, synthetic construct
GRKKRRQRRRPQKMILDCACKP

SEQ ID NO: 39 - protein, 22 aa, synthetic construct
GRKKRRQRRRPQPRQVPWPCKM

SEQ ID NO: 40 - protein, 22 aa, synthetic construct
GRKKRRQRRRPQDCMKLKIAPC

SEQ ID NO: 41 - protein, 10 aa, synthetic construct
PMCKCDILKA

SEQ ID NO: 42 - protein, 10 aa, synthetic construct
PMCKVPPWRQ

SEQ ID NO: 43 - protein, 7 aa, synthetic construct
CKVPPWR

SEQ ID NO: 44 - protein, 9 aa, synthetic construct
KVQLLPPWR